(19) United States
(12) Patent Application Publication
(10) Pub. No.: US 2016/0193249 A1
(43) Pub. Date: Jul. 7, 2016

(54) TREATMENT OF INFLAMMATORY DISEASES BY CARBON MATERIALS

(71) Applicants: WILLIAM MARSH RICE UNIVERSITY, Houston, TX (US); BAYLOR COLLEGE OF MEDICINE, Houston, TX (US)

(72) Inventors: James M. Tour, Bellaire, TX (US); Christine Beeton, Pearland, TX (US); Redwan U. Huq, Houston, TX (US); Taeko Inoue, Brownsville, TX (US); Robia G. Pautler, Pearland, TX (US); Errol L.G. Samuel, Houston, TX (US)

(21) Appl. No.: 14/915,858

(22) PCT Filed: Sep. 3, 2014
(86) PCT No.: PCT/US2014/053909

§ 371 Date: Mar. 1, 2016

Related U.S. Application Data
(60) Provisional application No. 61/873,046, filed on Sep. 3, 2013.

(51) Int. Cl. A61K 33/44; A61K 9/00; A61K 31/047

(52) U.S. Cl. CPC A61K 33/44; A61K 31/047; A61K 9/0092

(57) ABSTRACT

In some embodiments, the present disclosure pertains to methods of treating an inflammatory disease in a subject by administering a carbon material to the subject. In some embodiments, the carbon material selectively targets T cells in the subject. In some embodiments, the carbon material includes poly(ethylene glycol)-functionalized hydrophilic carbon clusters. In some embodiments, the administration of the carbon material to the subject reduces or inhibits T cell-mediated reactions in the subject. In some embodiments, the carbon material selectively targets T cells over other types of immune cells by preferential uptake into the T cells. In some embodiments, the carbon material reduces or inhibits proliferation of targeted T cells, reduces or inhibits cytokine production by targeted T cells, and reduces intracellular oxidant content in targeted T cells. In some embodiments, the present disclosure pertains to methods of modulating T cells ex-vivo by incubating the T cells with a carbon material.

### CROSS-REFERENCE TO RELATED APPLICATIONS

This application claims priority to U.S. Provisional Patent Application No. 61/873,046, filed on Sep. 3, 2013. The entirety of the aforementioned application is incorporated herein by reference.

### STATEMENT REGARDING FEDERALLY SPONSORED RESEARCH

This invention was made with government support under Grant No. W81XWH-12-1-0612, awarded by the U.S. Department of Defense; and Grant No. DK093802, awarded by the National Institutes of Health. The Government has certain rights in the invention.

### BACKGROUND

Current methods and therapeutic compositions for treating inflammatory diseases suffer from numerous limitations, including generalized immunosuppression that can in turn cause malignancies (e.g., cancer) and infections. As such, a need exists for improved methods and compositions for treating inflammatory diseases.

### SUMMARY

In some embodiments, the present disclosure pertains to methods of treating an inflammatory disease in a subject. In some embodiments, the method includes administering a carbon material to the subject. In some embodiments, the carbon material selectively targets T cells in the subject.

In some embodiments, the carbon material includes, without limitation, graphene quantum dots, graphene, graphene oxide, carbon black, activated carbon, carbon nanotubes, ultra-short single-walled carbon nanotubes (also referred to as hydrophilic carbon clusters or HCCs) and combinations thereof. In some embodiments, the carbon material has a serum half-life of between about 15 hours to about 40 hours.

In some embodiments, the carbon material has a length ranging from about 10 nm to about 100 nm. In some embodiments, the carbon material has a length ranging from about 10 nm to about 50 nm.

In some embodiments, the carbon material is oxidized. In some embodiments, the carbon material is functionalized with a plurality of functional groups. In some embodiments, the functional groups include, without limitation, polyethylene glycols, polypropylene glycols, poly(acrylic acid), polysaccharides, poly(alcohols), poly(vinyl alcohol), polyamines, polyethylene imines, poly(vinyl amines), ketones, esters, amides, carboxyl groups, oxides, hydroxyl groups, alkoxy groups, and combinations thereof. In some embodiments, the carbon material also includes one or more transport moieties.

In some embodiments, the carbon material includes ultra-short single-wall carbon nanotubes (i.e., HCCs). In some embodiments, the ultra-short single-wall carbon nanotubes are functionalized with a plurality of functional groups. In some embodiments, the ultra-short single-wall carbon nanotubes include poly(ethylene glycol)-functionalized ultra-short single-walled carbon nanotubes (also referred to as PEG-HCCs). In some embodiments, the ultra-short single-walled carbon nanotubes have lengths ranging from about 10 nm to about 100 nm, or from about 10 nm to about 50 nm.

In some embodiments, the carbon materials of the present disclosure are administered to a subject suffering from an inflammatory disease. In some embodiments, the inflammatory disease to be treated includes, without limitation, chronic inflammatory diseases, autoimmune diseases, T cell-mediated diseases, T cell-mediated autoimmune diseases, T cell-mediated inflammatory diseases, multiple sclerosis, rheumatoid arthritis, reactive arthritis, ankylosing spondylitis, systemic lupus erythematosus, glomerulonephritis, psoriasis, scleroderma, alopecia aerata, type 1 diabetes mellitus, celiac sprue disease, colitis, pernicious anemia, encephalomyelitis, vasculitis, thyroiditis, Addison's disease, Sjögren's syndrome, antiphospholipid syndrome, autoimmune cardiomyopathy, autoimmune hemolytic anemia, autoimmune hepatitis, autoimmune inner ear disease, autoimmune lymphoproliferative disorder, autoimmune peripheral neuropathy, pancreatitis, polyendocrine syndrome, thrombocytopenic purpura, uveitis, Behcet's disease, narcolepsy, myositis, polychondritis, asthma, chronic obstructive pulmonary disease, graft-versus-host disease, chronic graft rejection, and combinations thereof.

In some embodiments, the administering of the carbon material to the subject reduces or inhibits T cell-mediated reactions in the subject (e.g., T cell-mediated inflammatory reactions). In some embodiments, the carbon material selectively targets T cells over other types of immune cells.

In some embodiments, the carbon material selectively targets T cells by preferential uptake into the targeted T cells. In some embodiments, the carbon material reduces or inhibits proliferation of targeted T cells. In some embodiments, the carbon material reduces or inhibits cytokine production by targeted T cells. In some embodiments, the carbon material reduces or inhibits T cell signaling by targeted T cells. In some embodiments, the carbon material reduces intracellular oxidant content in targeted T cells. In some embodiments, the carbon material does not induce apoptosis in targeted T cells.

In some embodiments, the present disclosure pertains to methods of modulating T cells by incubating the T cells with a carbon material. In some embodiments, the method occurs ex-vivo.

### DESCRIPTION OF THE FIGURES

FIG. 1 provides a scheme of a method of treating an inflammatory disease (FIG. 1A) and a chemical structure of poly(ethylene glycol)-functionalized hydrophilic carbon clusters (PEG-HCCs) (FIG. 1B).

FIG. 2 shows that T cells selectively take up PEG-HCCs. FIG. 2A shows results demonstrating that PEG-HCCs were internalized by T cells. Rat splenocytes were incubated with 0.1 μg/ml of PEG-HCCs. The splenocytes were then washed and analyzed by flow cytometry (FCM), which demonstrated an increased signal from an anti-PEG antibody after cell permeabilization (particularly in CD3⁺ T cells). The results in FIG. 2A are representative of three experiments and indicative of PEG-HCC internalization. FIG. 2B shows results demonstrating the preferential uptake of PEG-HCCs by T cells over other immune cells (i.e., splenic immune cells) in vitro, as determined by FCM analysis (n=3). FIG. 2C shows the pharmacokinetics of PEG-HCCs in rat serum, as determined by a single subcutaneous injection of 2 mg/kg of PEG-HCCs (left panel). Blood was collected at the indicated times and nanoparticle concentration was measured by an enzyme-linked immunosorbent assay (ELISA) (n=5-6 rats per time point). Data fit to a single exponential decay to calculate circulating half-life of ˜25 h (right panel). FIG. 2D shows results demonstrating the preferential uptake of PEG-HCCs by T cells over macrophages and T cells in vivo, as analyzed using FCM. Rats were injected with 2 mg/kg of PEG-HCCs. Splenocytes were then isolated after 24 hours. The results are consistent with in vitro results in FIG. 2B (n=3 rats). ***P<0.001, ****P<0.0001. Data are expressed as means±s.e.m. FIG. 2E outlines the gating strategy used for determining cellular uptake of PEG-HCCs by immune cells via flow cytometry and identifying uptake of PEG-HCCs.

FIG. 3 shows that PEG-HCCs enter T cells mainly via endocytosis and are gradually lost. FIG. 3A provides data indicating that the T cell uptake of PEG-HCCs (as analyzed by FCM) is diminished under endocytosis-inhibiting conditions (4° C.), as compared to physiological conditions (37° C.) (n=3). FIG. 3B shows data relating to the kinetics of nanoparticle internalization in splenic T cells incubated for the indicated times with 0.1 μg/ml of PEG-HCCs prior to FCM analysis (n=3). FIG. 3C shows data relating to the kinetics of loss of nanoparticles in splenic T cells. The cells were incubated for 30 minutes with 0.1 μg/ml of PEG-HCCs. The cells were then washed and analyzed by FCM after the indicated times (n=3). *P<0.05, **P<0.01, ****P<0.0001. Data are expressed as means±s.e.m.

FIG. 4 demonstrates that PEG-HCCs suppress T cell activity upon internalization. FIG. 4A shows that the proliferation of primary GFP-transduced ovalbumin-specific rat T cells (CD4⁺CCR7⁻CD45RC⁻Kv1.3^{high}), stimulated with ovalbumin and as measured by [³H] thymidine incorporation, is decreased after incubation with the indicated concentrations of PEG-HCCs (n=3). FIG. 4B shows that the proliferation of stimulated T cells remains unaltered if cells are washed off excess PEG-HCCs after incubation, indicating the reduction in proliferation requires nanoparticle internalization. Proliferation is rescued if T cells are incubated with PEG-HCCs, washed and then stimulated after 6 hours, showing good agreement with kinetics of nanoparticle loss, and suggesting that PEG-HCC effect on T proliferation is reversible (n=3). FIG. 4C provides data relating to the quantification of cell death in T cells that are unstimulated, stimulated, and incubated with PEG-HCCs prior to stimulation, or stimulated and treated with staurosporine. The cells were analyzed by 7-aminoactinomycin-D (7-AAD) staining and FCM (n=4). FIG. 4D shows that the production of pro-inflammatory cytokines (IL-2 and IFN-γ, as analyzed by FCM) is reduced in T cells that are incubated with the indicated concentrations of PEG-HCCs and stimulated (n=6). *P<0.05, **P<0.01, ****P<0.0001. Data are expressed as means±s.e.m.

FIG. 5 shows that PEG alone is not sufficient to decrease the proliferation of T cells. Proliferation of stimulated ovalbumin-specific rat T cells, as measured by [³H] thymidine incorporation, is not affected by the indicated concentrations of PEG-5000, as compared to PEG-HCCs (n=3).

FIG. 6 shows that the failure of macrophages to internalize PEG-HCCs leaves key macrophage functions unaltered. FIG. 6A shows T cell migration across transwell filters towards supernatant collected from primary peritoneal rat macrophages stimulated with lipopolysaccharides (LPS). T cell migration remains unchanged if macrophages are incubated with PEG-HCCs prior to stimulation (green), indicating that PEG-HCCs do not affect chemo-attractant production by macrophages. Migration of T cells, incubated with PEG-HCCs (blue), also remain unchanged, suggesting nanoparticles do not affect T cells that are not stimulated (n=3). FIG. 6B shows data relating to the phagocytosis of macrophages (as quantified by the uptake of zymosan A bioparticles using confocal microscopy) after incubation with the indicated concentrations of PEG-HCCs or Fe₃O₄ nanoparticles (n=3). Corresponding images of Alexa Fluor 488-conjugated bioparticles (green) and macrophage nuclei stained with DAPI (blue) are shown on the lower panel. The scale bars are 5 μm. FIG. 6C shows antigen processing and presentation of macrophages gauged by the proliferation of ovalbumin-specific T cells stimulated by macrophages pre-incubated with ovalbumin (stimulated). Incubating macrophages with PEG-HCCs prior to adding T cells does not affect T cell proliferation (green), whereas incubating T cells with PEG-HCCs (blue) decreases their proliferation (n=3). *P<0.05, **P<0.01, ***P<0.001, ****P<0.0001. Data are expressed as means±s.e.m.

FIG. 7 shows that PEG-HCCs do not decrease the proliferation of T cells that have not been stimulated. Proliferation of resting ovalbumin-specific rat T cells, as measured by [³H] thymidine incorporation, is not affected by the indicated concentrations of PEG-HCCs, suggesting that an increase in intracellular SO during T cell activation is necessary for PEG-HCCs to alter cellular function (n=3).

FIG. 8 shows that the administration of PEG-HCCs suppresses T cell-mediated inflammation and ameliorates experimental autoimmune encephalomyelitis (EAE). FIG. 8A shows that a single subcutaneous injection of 2 mg/kg of PEG-HCCs reduces an active delayed-type hypersensitivity response elicited against ovalbumin in the ears of rats, either at immunization or challenge, compared to PBS (Vehicle) treatment. Ear swelling was measured 24 hours after challenge (n=5 rats per group). FIG. 8B shows clinical scores of rats with EAE, treated every three days with PBS (Vehicle) or PEG-HCCs (2 mg/kg) subcutaneously at the onset of disease signs (n=6 rats per group). FIG. 8C shows the histological analysis of spinal cords collected from rats with EAE at the peak of disease, stained with hematoxylin and eosin, and quantified blindly for degree of immune infiltration from eight random fields of view (n=3 rats per group). Scale bars, 100 μm *P<0.05, **P<0.01, ***P<0.001. Data are expressed as means±s.e.m.

FIG. 9 shows that PEG-HCCs cross the plasma membrane of human T cells and suppress T cell activity upon internalization. FIG. 9A shows a flow cytometry histogram of the relative cell numbers of human mononuclear blood cells incubated with PEG-HCCs.

Mononuclear cells were incubated with 0.01 μg/ml of PEG-HCCs for 10 minutes and stained with an anti-CD3 antibody to detect T cells. An anti-PEG antibody was used to detect PEG-HCCs on intact cells (red) or after cell permeabilization (blue). Untreated cells are shown as a black dotted line. FIG. 9B shows that the proliferation of primary human T cells, stimulated by phytohemagglutinin and measured by [³H] thymidine incorporation, is decreased after incubation with the indicated concentrations of PEG-HCCs (n=3 donors). ***P<0.001, ****P<0.0001. Data are expressed as means±s.e.m.

FIG. 10 shows that PEG-HCCs reduce the number of lesions to the blood-brain barrier in an active acute model of multiple sclerosis in rats. The number of Gd³⁺ enhancing lesions to the blood-brain barrier (BBB, yellow arrows) is reduced in a rat model of active acute EAE during treatment with PEG-HCCs (FIG. 10B) compared with treatment with vehicle (FIG. 10A). In the PEG-HCC-treated rats (FIG. 10B), only two small lesions were observed. In vehicle-treated animals (FIG. 10A), the lesions were numerous. FIG. 10C provides a quantification of the number of BBB lesions. p=0.08 with n=3 rats per group.

FIG. 11 shows that PEG-HCCs reduce disease severity in pristane-induced arthritis, a rat model of inflammatory arthritis. The diagram shows the mean clinical score of the PEG-HCCs-treated rats (n=8 rats) compared to rats treated with PBS (Vehicle) (n=15 rats), every four days starting at the onset of disease. Clinical scoring included 5 points per large red and swollen joint (wrist, ankle) and 1 point per small red and swollen joint (mid-foot, digit, knuckle). **p<0.01, ***p<0.001.

FIG. 12 shows that PEG-HCCs follow a trend in reducing clinical scores during the relapsing phase of relapsing experimental autoimmune encephalomyelitis (R-EAE) in a small pilot trial. R-EAE was induced by immunizing DA rats against rat spinal cord in emulsion with complete Freund's adjuvant. Treatment with PEG-HCCs or PBS (vehicle) began at the time of immunization. Clinical scoring scales included: 0, no disease; 1, limp tail; 2: mild paraparesis, ataxia; 3: moderate paraparesis; 4, complete hind limb paralysis; 5, 4+incontinence; and 6, moribund, requires euthanasia. Relapses are defined as a change in at least a full score point for at least 2 consecutive observations.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Inflammatory diseases (e.g., multiple sclerosis, rheumatoid arthritis, type-1 diabetes mellitus, asthma, and vasculitis) affect millions of people worldwide and cause a significant reduction in quality of life and even death. T cells play major roles in those diseases by entering inflamed tissues and producing large amounts of chemokines and cytokines.

Moreover, excessive quantities of oxidants have been implicated in the pathogenesis of T cell-mediated inflammatory diseases. In particular, low levels of oxidants, such as intracellular reactive oxygen species (ROS), are produced in response to T cell receptor stimulation. Such oxidants can in turn act as second messengers during T cell activation.

For instance, during multiple sclerosis (MS), excess superoxide (SO) and hydroxyl radicals are produced in the CNS by microglia, astrocytes, and infiltrating immune cells. SO plays an important role in the activation of T cells through the T cell receptor. In addition, hydroxyl radicals directly damage the myelin during MS.

Current therapies for most inflammatory diseases (e.g., autoimmune diseases) involve the administration of generalized immunosuppressants. Antioxidants that target oxidants have also been utilized as an alternate route of therapy for T cell-mediated inflammatory diseases. However, such treatments have numerous limitations.

For instance, generalized immunosuppressants are associated with deleterious side effects, such as infections and malignancies. Moreover, endogenous and dietary antioxidants have shown only modest clinical efficacy. Such limited clinical efficacies can be attributed to poor selectivity for radical annihilation, rapid inactivation, limited stoichiometric capacity, and dependence on other detoxifying molecules. In addition, dietary antioxidants require the administration of high dosages, which increase mortality. For instance, administration of high doses or long-term use of broad antioxidants, such as Vitamin E, is toxic.

Accordingly, non-toxic agents that act as potent antioxidants have been assessed as therapeutic options for the treatment of various inflammatory diseases, such as MS. For instance, dimethylfumarate, a nuclear factor erythroid 2-related factor 2 (Nrf-2) activator, is an oral medication taken 2-3 times per day that activates multiple antioxidant pathways through the antioxidant response element. Improvements in contrast enhanced MRI have been reported in MS patients treated with dimethylfumarate with minimal side effects that include gastro-intestinal disturbances or tingling.

While encouraging, the fact that dimethylfumarate impacts a large array of ROS could have significant long term health effects because normal levels of ROS are necessary in many normal physiological processes, including long term potentiation and vascular tone. In addition, expression levels of Nrf-2 decrease with age, suggesting a reduction in efficacy of Nrf-2 activators in aging patients. Moreover, dimethylfumarate induces the apoptosis of activated human T cells. Furthermore, the administration of dimethylfumarate results in a reduction in circulating T cell numbers.

Therefore, a need exists for more effective methods and compositions for treating inflammatory diseases (e.g., T cell-mediated autoimmune or inflammatory diseases) without causing generalized immunosuppression or cell death. The present disclosure addresses this need.

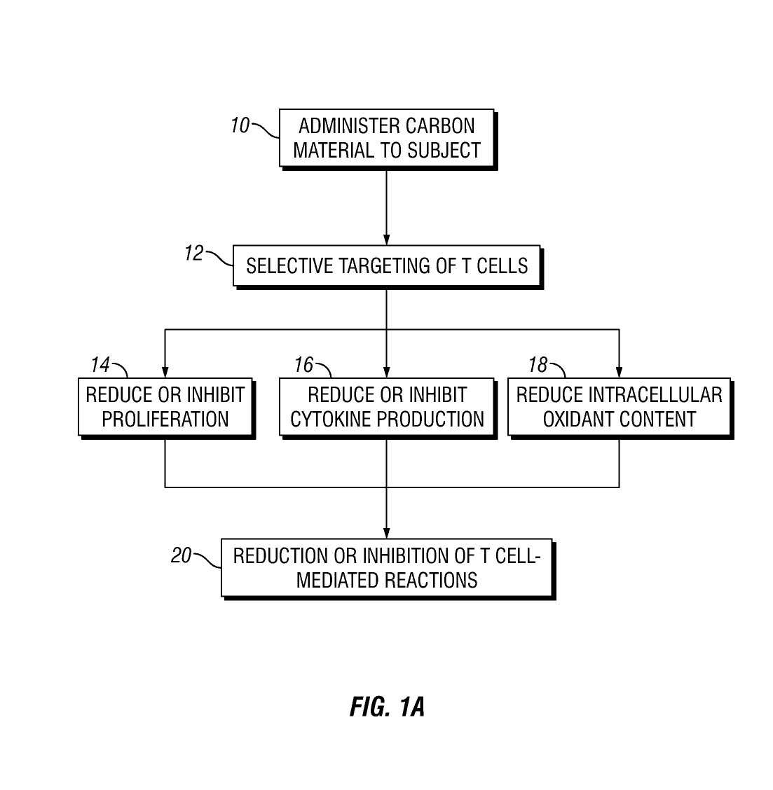

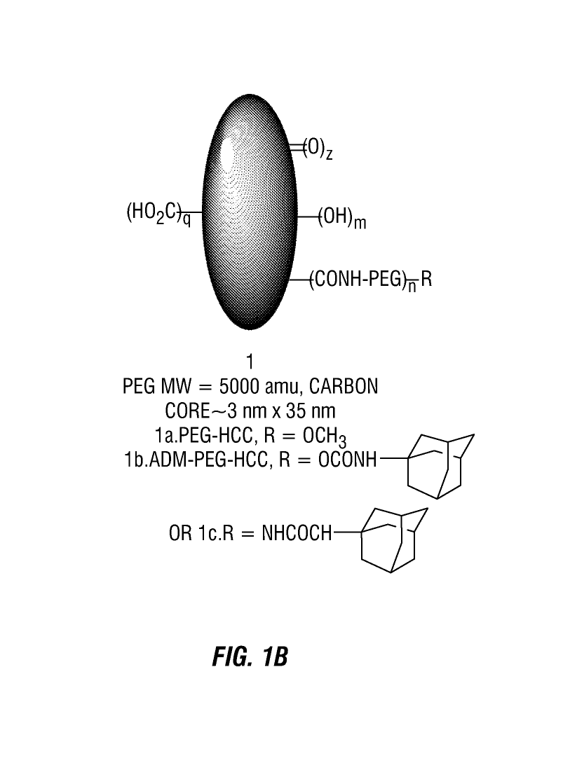

In some embodiments illustrated in FIG. 1A, the present disclosure pertains to methods of treating an inflammatory disease by administering a carbon material to the subject (step 10). In some embodiments, the administered carbon material selectively targets T cells in the subject (step 12). In some embodiments, the carbon material effects targeted T cells by reducing or inhibiting targeted T cell proliferation (step 14), reducing or inhibiting cytokine production by targeted T cells (step 16), or reducing the intracellular oxidant content of the targeted T cells (step 18). Such effects can in turn reduce or inhibit T cell-mediated reactions in the subject (step 20).

As set forth in more detail herein, the methods of the present disclosure can have various embodiments. For instance, various carbon materials may be administered by different modes to various subjects in order to treat a variety of inflammatory diseases. Moreover, the carbon materials of the present disclosure may selectively target and affect numerous types of T cells in various manners.

Carbon Materials

The methods of the present disclosure may utilize various types of carbon materials to treat inflammatory diseases. In some embodiments, suitable carbon materials include carbon materials that are capable of selectively targeting T cells. In some embodiments, suitable carbon materials include carbon materials that are capable of reducing or inhibiting T cell-mediated reactions (e.g., T cell-mediated inflammatory reactions).

In some embodiments, the carbon materials of the present disclosure may have properties that make them bio-available. For instance, in some embodiments, the carbon materials of the present disclosure may be hydrophilic (i.e., water soluble). In some embodiments, the carbon materials of the present disclosure may have both hydrophilic portions and hydrophobic portions. For instance, in some embodiments, the carbon materials of the present disclosure may have a hydrophilic domain (e.g, a hydrophilic surface) and a hydrophobic domain (e.g., a hydrophobic cavity). In some embodiments, the carbon material is in the form of aqueous or saline solutions.

In some embodiments, the carbon materials of the present disclosure have a serum half-life of between about 15 hours to about 40 hours. In some embodiments, the carbon materials of the present disclosure have a serum half-life of about 25 hours. In some embodiments, the carbon materials of the present disclosure have a serum half-life of between about 15 hours to about 40 hours when administered subcutaneously to a subject.

In some embodiments, the carbon materials of the present disclosure are in the form of a nanomaterial. For instance, in some embodiments, the carbon materials of the present disclosure are in the form of nanoparticles. In some embodiments, the carbon materials of the present disclosure have diameters ranging from about 1 nm to about 10 nm. In some embodiments, the carbon materials of the present disclosure have diameters of about 5 nm. In some embodiments, the carbon materials of the present disclosure have diameters of about 1 nm to about 2 nm.

In some embodiments, the carbon materials of the present disclosure have lengths ranging from about 10 nm to about 100 nm. In some embodiments, the carbon materials of the present disclosure have lengths ranging from about 30 nm to about 100 nm. In some embodiments, the carbon materials of the present disclosure have lengths ranging from about 10 nm to about 80 nm. In some embodiments, the carbon materials of the present disclosure have lengths ranging from about 10 nm to about 50 nm. In some embodiments, the carbon materials of the present disclosure have lengths ranging from about 10 nm to about 20 nm. In some embodiments, the carbon materials of the present disclosure have lengths of about 40 nm. In some embodiments, the carbon materials of the present disclosure include carbon nanoparticles that are about 30 nm to about 40 nm long, and approximately 1-2 nm wide. In some embodiments, the carbon materials of the present disclosure include carbon nanoparticles that are about 35 nm long and approximately 3 nm wide.

In some embodiments, the carbon materials of the present disclosure may not be associated with additional materials. For instance, in some embodiments, the carbon materials of the present disclosure are not associated with active pharmaceutical ingredients (e.g., active agents or drugs). In some embodiments, the carbon materials of the present disclosure are not associated with metals. In some embodiments, the carbon materials of the present disclosure may only be associated with undetectable or trace amounts of metals.

In some embodiments, the carbon materials of the present disclosure may be modified in various ways. For instance, in some embodiments, the carbon materials of the present disclosure are oxidized. In some embodiments, the carbon materials of the present disclosure are functionalized with a plurality of functional groups. In some embodiments, the functional groups promote the uptake of the carbon materials by T cells, and inhibit the uptake of the carbon materials by other cells, such as B cells, macrophages, dendritic cells, natural killer (NK) cells, natural killer T cells (NKT), and neutrophils. In some embodiments, the functional groups include, without limitation, polyethylene glycols, polypropylene glycols, poly(acrylic acid), polysaccharides, poly(alcohols), poly(vinyl alcohol), polyamines, polyethylene imines, poly(vinyl amines), ketone, esters, amides, carboxyl groups, oxides, hydroxyl groups, alkoxy groups, and combinations thereof.

In some embodiments, the functional groups include polyethylene glycols (PEGs). In some embodiments, the polyethylene glycols have molecular weights that range from about 5,000 atomic mass units (PEG-5000) to about 50 atomic mass units (PEG-50). In some embodiments, the polyethylene glycols have molecular weights that range from about 500 atomic mass units (PEG-500) to about 50 atomic mass units (PEG-50). In some embodiments, the polyethylene glycols include, without limitation, PEG-5000, PEG-500, PEG-100, PEG-50, and combinations thereof.

In some embodiments, the carbon materials of the present disclosure include one or more transport moieties. In some embodiments, the transport moieties assist in the transport of the carbon materials through various biological barriers, such as the blood-brain barrier or blood-spinal cord barrier. In some embodiments, transport moieties may also assist in recognition of certain cell types, such T cells. In some embodiments, the transport moieties may include, without limitation, adamantane moieties (ADM), dimethyladamantane moieties, lipophilic moieties, small molecules, cannabinoids, epi-cannabinoids, peptides, saccharides, and combinations thereof. In some embodiments, transport moieties may include enantiomers or diastereomers of cannabinoids.

In some embodiments, the transport moieties may be directly associated with carbon materials. In some embodiments, the transport moieties may be associated with functional groups that are directly associated with carbon materials. In some embodiments, the transport moieties may be attached to the terminal of functional groups (e.g., ADM moieties attached to the terminal end of PEG moieties).

In some embodiments, the carbon materials of the present disclosure may be associated with one or more surfactants. For instance, in some embodiments, the carbon materials are surfactant wrapped. In some embodiments, the carbon materials are pluronic wrapped.

In some embodiments, the serum half-life of the carbon materials of the present disclosure can be further extended by the modification of functional groups that are associated with the carbon materials. For instance, in some embodiments, the serum half-life of the carbon materials can be extended by extending the length, density, or branching of the functional groups associated with the carbon materials (e.g., PEG functional groups). In some embodiments, the serum half-life of the carbon materials can be extended by increasing the number of transport moieties associated with the carbon materials (e.g., ADM moieties attached to the terminal of PEG moieties).

In some embodiments, the carbon materials of the present disclosure can include, without limitation, graphene quantum dots, graphene, graphene oxide, carbon black, activated carbon, carbon nanotubes, ultra-short single-walled carbon nanotubes (also referred to as hydrophilic carbon clusters or HCCs), and combinations thereof.

In some embodiments, the aforementioned carbon materials may be functionalized with a plurality of functional groups, as previously described. In some embodiments, the aforementioned carbon materials may be associated with one or more transport moieties, as previously described. In some embodiments, the aforementioned carbon materials may be poly(ethylene glycol)-functionalized (PEG-functionalized) or further adamantyl (ADM) functionalized.

In some embodiments, the carbon materials of the present disclosure include carbon nanotubes. In some embodiments, the carbon nanotubes include, without limitation, single-walled carbon nanotubes, ultra-short single-walled carbon nanotubes, multi-walled carbon nanotubes, double-walled carbon nanotubes, and combinations thereof. In some embodiments, the carbon nanotubes may be functionalized with a plurality of functional groups (as previously described). In some embodiments, the carbon nanotubes may be oxidized.

In some embodiments, the carbon materials of the present disclosure include ultra-short single-walled carbon nanotubes (US-SWNTs). US-SWNTs are also referred to as hydrophilic carbon clusters (HCCs). In some embodiments, ultra-short single-walled carbon nanotubes are functionalized with a plurality of functional groups (as previously described). In some embodiments shown in FIG. 1B, the carbon materials of the present disclosure include poly(ethylene glycol)-functionalized ultra-short single-walled carbon nanotubes (also referred to as PEG-HCCs). In some embodiments, the PEG-HCCs may also be associated with one or more transport moieties, such as ADM (also referred to as ADM-PEG-HCCs).

In some embodiments, the carbon materials of the present disclosure include ultra-short single-walled carbon nanotubes with lengths that range from about 10 nm to about 100 nm. In some embodiments, the ultra-short single-walled carbon nanotubes have lengths that range from about 30 nm to about 100 nm. In some embodiments, the ultra-short single-walled carbon nanotubes have lengths that range from about 10 nm to about 80 nm. In some embodiments, the ultra-short single-walled carbon nanotubes have lengths that range from about 10 nm to about 50 nm. In some embodiments, the ultra-short single-walled carbon nanotubes have lengths that range from about 10 nm to about 20 nm. In some embodiments, the ultra-short single-walled carbon nanotubes have lengths of about 40 nm. In some embodiments, the ultra-short single-walled carbon nanotubes have lengths of about 35 nm.

In some embodiments, the ultra-short single-walled carbon nanotubes are not associated with metals. In some embodiments, the ultra-short single-walled carbon nanotubes are in dispersed form. In some embodiments, the ultra-short single-walled carbon nanotubes are water soluble and hydrophilic. In some embodiments, ultra-short single-walled carbon nanotubes are prepared by exposing single-walled carbon nanotubes to superacids, such as fuming sulfuric acid and nitric acid. Examples of such methods of preparing ultra-short single-walled carbon nanotubes are disclosed in U.S. Pat. No. 8,313,724; U.S. Pat. App. Pub. Nos. 2012/0302816 and 2009/0170768; and PCT App. Nos. PCT/US2012/035267, PCT/US2012/035244, and PCT/US2013/032502.

Additional examples of ultra-short single-walled carbon nanotubes and methods of making them are disclosed in the following articles and applications: Berlin et al., ACS Nano 2010, 4, 4621-4636; Lucente-Schultz et al., J. Am. Chem. Soc. 2009, 131, 3934-3941; Chen et al., J. Am. Chem. Soc. 2006, 128, 10568-10571; Stephenson, et al., Chem. Mater. 2007, 19, 3491-3498; Price et al., Chem. Mater. 2009, 21, 3917-3923; PCT/US2008/078776; and PCT/US2010/054321.

In some embodiments, the carbon materials of the present disclosure include graphene quantum dots. In some embodiments, the graphene quantum dots include, without limitation, oxidized graphene quantum dots, graphene quantum dots derived from coal, graphene quantum dots derived from coke, graphene quantum dots derived from asphalt, oxidized graphene quantum dots derived from coal, and combinations thereof. In some embodiments, the graphene quantum dots are functionalized with a plurality of functional groups (as previously described). In some embodiments, the graphene quantum dots include polyethylene glycol-functionalized graphene quantum dots. In some embodiments, graphene quantum dots are prepared by methods disclosed in PCT App. No. PCT/US2014/036604.

In some embodiments, the carbon materials of the present disclosure include activated carbons. In some embodiments, activated carbons include oxidized activated carbon. In some embodiments, the activated carbons are functionalized with a plurality of functional groups (as previously described). In some embodiments, the activated carbons include polyethylene glycol-functionalized activated carbons.

In some embodiments, the carbon materials of the present disclosure include carbon black. In some embodiments, the carbon black includes oxidized carbon black. In some embodiments, the carbon black is functionalized with a plurality of functional groups (as previously described). In some embodiments, the carbon black includes polyethylene glycol-functionalized carbon black.

Administration of Carbon Materials to Subjects

The carbon materials of the present disclosure can be administered to subjects by various methods. For instance, in some embodiments, the carbon materials of the present disclosure can be administered by oral administration (including gavage), inhalation, subcutaneous administration (sub-q), topical administration, transdermal administration, intra-articular administration, intravenous administration (I.V.), intraperitoneal administration (I.P.), intramuscular administration (I.M.), intrathecal injection, sub-lingual administration, intranasal administration, and combinations of such modes. In some embodiments, the carbon materials of the present disclosure can be administered by topical application (e.g, transderm, ointments, creams, salves, eye drops, and the like).

In some embodiments, the carbon materials of the present disclosure can be administered by intravenous administration. In some embodiments, the carbon materials of the present disclosure can be administered by transdermal administration. In some embodiments, the carbon materials of the present disclosure can be administered by transdermal administration through the use of patches that contain the carbon materials.

In some embodiments, the carbon materials of the present disclosure can be administered by intra-articular administration for the treatment of arthritis. In some embodiments, the carbon materials of the present disclosure can be administered by intranasal administration. In some embodiments, the intranasal administration leads to the delivery of the carbon materials into the airways of a subject (e.g., lungs and trachea). In some embodiments, the intranasal administration leads to the delivery of the carbon materials into the central nervous system of a subject (e.g., the brain). In some embodiments, the carbon materials of the present disclosure can be administered by intranasal administration for delivery into the central nervous system of a subject for the treatment of multiple sclerosis.

In some embodiments, the administration of carbon materials may occur selectively at a desired site. For instance, in some embodiments, the carbon materials of the present disclosure may be administered to the lungs or central nervous system of a subject. Additional modes of administration can also be envisioned.

The administering of the carbon materials of the present disclosure can occur for various periods of time. For instance, in some embodiments, the administering of the carbon material can include, without limitation, hourly administration, daily administration, weekly administration, monthly administration, and combinations thereof.

In some embodiments, the administering of the carbon material includes daily administration. In some embodiments, the daily administration lasts from about 3 days to about 3 months. In some embodiments, the daily administration may include one or more carbon material administrations per day. For instance, in some embodiments, the daily administration can include from about 1 carbon material administration per day to about 5 carbon material administrations per day.

The carbon materials of the present disclosure may also be administered at various dosages. For instance, in some embodiments, carbon material administration occurs at dosages that range from about 1 mg/kg of the subject's weight to about 5 mg/kg of the subject's weight. In some embodiments, carbon material administration occurs at about 2 mg/kg of the subject's weight.

Subjects

The carbon materials of the present disclosure may be administered to various subjects. For instance, in some embodiments, the subject is a human being. In some embodiments, the subject may be a non-human animal, such as mice, rats, other rodents, or larger mammals, such as dogs, monkeys, pigs, cattle and horses. In some embodiments, the subject may be a mammal, such as a dog.

In some embodiments, the subject may be suffering from an inflammatory disease. In some embodiments, the subject suffering from an inflammatory disease is a mammal. In some embodiments, the subject suffering from an inflammatory disease is a human being. In some embodiments, the subject suffering from an inflammatory disease is a dog or another animal.

Treatment of Inflammatory Diseases

The carbon materials of the present disclosure may be utilized to treat various inflammatory diseases in subjects. For instance, in some embodiments, the inflammatory diseases that can be treated by the carbon materials of the present disclosure can include, without limitation, chronic inflammatory diseases, autoimmune diseases, T cell-mediated diseases, T cell-mediated autoimmune diseases, T cell-mediated inflammatory diseases, multiple sclerosis, rheumatoid arthritis, reactive arthritis, ankylosing spondylitis, systemic lupus erythematosus, glomerulonephritis, psoriasis, scleroderma, alopecia aerata, type 1 diabetes mellitus, celiac sprue disease, colitis, pernicious anemia, encephalomyelitis, vasculitis, thyroiditis, Addison's disease, Sjögren's syndrome, antiphospholipid syndrome, autoimmune cardiomyopathy, autoimmune hemolytic anemia, autoimmune hepatitis, autoimmune inner ear disease, autoimmune lymphoproliferative disorder, autoimmune peripheral neuropathy, pancreatitis, polyendocrine syndrome, thrombocytopenic purpura, uveitis, Behcet's disease, narcolepsy, myositis, polychondritis, asthma, chronic obstructive pulmonary disease, graft-versus-host disease, chronic graft rejection, and combinations thereof.

The carbon materials of the present disclosure can be utilized to treat various symptoms of inflammatory diseases. For instance, in some embodiments, the administering of a carbon material to a subject can decrease inflammation associated with an inflammatory disease in the subject (e.g., swollen joints associated with an inflammatory disease, such as arthritis). In some embodiments, the administering of a carbon material to a subject can reduce the number of lesions associated with an inflammatory disease in the subject. In some embodiments, the number of lesions is reduced by about 10% to about 100% in the subject. In some embodiments, the number of lesions is reduced by about 10% to about 50% in the subject. In some embodiments, the number of lesions is reduced by about 33% in the subject. In some embodiments, the lesions are eliminated in the subject. In some embodiments, the lesions are associated with multiple sclerosis. In some embodiments, the lesions are near the blood-brain barrier.

Without being bound by theory, it is envisioned that the carbon materials of the present disclosure can treat inflammatory diseases by various mechanisms. For instance, in some embodiments, the administering of a carbon material to a subject can reduce or inhibit T cell-mediated reactions in a subject (e.g., T cell-mediated inflammatory reactions). In some embodiments, the administering of a carbon material to a subject can prevent, delay, reduce or inhibit delayed type hypersensitivity (DTH) reactions associated with an inflammatory disease in a subject.

Effect of Carbon Materials on Targeted T Cells

Without being bound by further theory, the carbon materials of the present disclosure can treat inflammatory diseases by various cellular mechanisms. For instance, in some embodiments, the carbon materials of the present disclosure can selectively target T cells over other types of immune cells. In some embodiments, other types of immune cells that are not targeted by the carbon materials of the present disclosure can include, without limitation, macrophages, B cells, granulocytes, dendritic cells, neutrophils, natural killer (NK) cells, NKT cells and combinations thereof.

In some embodiments, the carbon materials of the present disclosure selectively target T cells over B cells, macrophages, NK cells, NKT cells, dendritic cells, and neutrophils. In some embodiments, the carbon materials of the present disclosure selectively target T cells without having any effect on macrophages. For instance, in some embodiments, the carbon materials of the present disclosure affect the activity of T cells without affecting the activity of macrophages (e.g., phagocytosis, antigen processing and presentation, or chemo-attraction by macrophages).

The carbon materials of the present disclosure can selectively target various types of T cells. For instance, in some embodiments, the carbon materials of the present disclosure selectively target effector-memory T cells (T_{EM} cells).

The carbon materials of the present disclosure can selectively target T cells by various mechanisms. For instance, in some embodiments, the carbon materials of the present disclosure selectively target T cells by the preferential uptake of the carbon materials into the targeted T cells. In some embodiments, targeted T cells may display a higher uptake capacity for the carbon material than other immune cells. In some embodiments, targeted T cells have an uptake capacity for the carbon material that is about 10% to about 100% higher than the uptake capacity of other immune cells for the carbon material. In some embodiments, targeted T cells have an uptake capacity for the carbon material that is about 10% to about 20% higher than the uptake capacity of other immune cells for the carbon material. In some embodiments, targeted T cells have an uptake capacity for the carbon material that is about 10% to about 50% higher than the uptake capacity of other immune cells for the carbon material.

The carbon materials of the present disclosure can also enter targeted T cells by various mechanisms. For instance, in some embodiments, the carbon materials of the present disclosure enter targeted T cells by crossing the plasma membrane of the T cells. In some embodiments, the carbon materials of the present disclosure enter targeted T cells by endocytosis.

Without being bound by further theory, it is envisioned that the carbon materials of the present disclosure can have various effects on the targeted T cells. For instance, in some embodiments, the carbon materials of the present disclosure reduce or inhibit the proliferation of targeted T cells. In some embodiments, the carbon materials of the present disclosure reduce targeted T cell proliferation by about 10% to about 100%. In some embodiments, the carbon materials of the present disclosure reduce targeted T cell proliferation by about 40% to about 100%. In some embodiments, the carbon materials of the present disclosure reduce targeted T cell proliferation by about 50%.

In some embodiments, the carbon materials of the present disclosure reduce or inhibit cytokine production by targeted T cells. For instance, in some embodiments, the carbon materials of the present disclosure reduce or inhibit cytokine production in targeted T cells by about 10% to about 80%. In some embodiments, the carbon materials of the present disclosure reduce or inhibit cytokine production in targeted T cells by about 20% to about 40%. In some embodiments, the carbon materials of the present disclosure reduce or inhibit cytokine production by the T cells by about 30%.

In some embodiments, the carbon materials of the present disclosure reduce or inhibit the production of pro-inflammatory cytokines in targeted T cells. In some embodiments, the pro-inflammatory cytokines include, without limitation, interleukins, interferons, and combinations thereof. In some embodiments, the pro-inflammatory cytokines include, without limitation, interleukin (IL)-2 and interferon (IFN)-γ.

In some embodiments, the carbon material reduces or inhibits T cell signaling by targeted T cells. In some embodiments, T cell signaling is reduced or inhibited as a result of a reduction or inhibition of cytokine production.

In some embodiments, the carbon materials of the present disclosure reduce the intracellular oxidant content of targeted T cells. In some embodiments, the reduced oxidants can include, without limitation, superoxide (SO), hydroxyl radicals, reactive oxygen species (ROS), and combinations thereof. In some embodiments, the carbon materials of the present disclosure reduce intracellular oxidant contents by scavenging the oxidants. In some embodiments, the carbon materials of the present disclosure reduce intracellular oxidant contents by catalytically converting the oxidants. In some embodiments, the carbon materials of the present disclosure have no substantial effects on the oxidant contents of other immune cells.

In some embodiments, the carbon materials of the present disclosure affect the activity of targeted T cells in a reversible manner. In some embodiments, the carbon materials of the present disclosure affect the activity of targeted T cells in a dose-dependent manner. In some embodiments, the carbon materials of the present disclosure affect the activity of targeted T cells without affecting the viability of the targeted T cells. For instance, in some embodiments, the carbon materials of the present disclosure affect the activity of targeted T cells without inducing apoptosis in targeted T cells. In some embodiments, the carbon materials of the present disclosure cause the death of less than 10% of the targeted T cells.

Modulation of T Cells

In some embodiments, the present disclosure pertains to methods of modulating T cells by incubating the T cells with a carbon material. In some embodiments, the method occurs ex-vivo. In some embodiments, the method occurs ex-vivo in the presence of other types of immune cells (as previously described). In some embodiments, the method occurs in vitro. In some embodiments, the carbon material selectively targets T cells over other types of immune cells (as previously described). In some embodiments, the carbon material selectively targets T cells by preferential uptake into the T cells (as previously described).

In some embodiments, the carbon material reduces or inhibits T-cell mediated reactions (as previously described). In some embodiments, the carbon material reduces or inhibits proliferation of targeted T cells (as previously described). In some embodiments, the carbon material reduces or inhibits cytokine production by targeted T cells (as previously described). In some embodiments, the carbon material reduces or inhibits T cell signaling by targeted T cells (as previously described). In some embodiments, the carbon material reduces intracellular oxidant content in targeted T cells (as previously described). In some embodiments, the carbon material does not induce apoptosis in targeted T cells (as previously described).

Various carbon materials may be utilized to modulate T cells. Suitable carbon materials were described previously. In some embodiments, the carbon materials include ultra-short single-wall carbon nanotubes. In some embodiments, the ultra-short single-wall carbon nanotubes are functionalized with a plurality of functional groups. In some embodiments, the ultra-short single-wall carbon nanotubes include poly(ethylene glycol)-functionalized ultra-short single-walled carbon nanotubes.

Advantages

The present disclosure provides improved methods and carbon materials for treating various types of inflammatory conditions without causing generalized immunosuppression. Moreover, the carbon materials of the present disclosure can specifically target T cells in a reversible and non-toxic manner. As such, the methods and carbon materials of the present disclosure offer significant advantages over existing methods and compositions of treating inflammatory diseases. For instance, the methods and carbon materials of the present disclosure can treat various types of inflammatory diseases without the side-effects that are associated with conventional treatment methods, including the development of malignancies (e.g., cancer) and infections.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1

### Preferential Uptake of PEG-HCCs by T Cells

In this Example, Applicants show that poly(ethylene)-glycol-functionalized hydrophilic carbon clusters (PEG-HCCs) preferentially enter T cells over macrophages, B cells, NK cells, NKT cells, dendritic cells and neutrophils. Applicants also apply this property to attenuate the activity of disease-associated T cells, and ameliorate experimental autoimmune encephalomyelitis (EAE) and pristane-induced arthritis (animal models of multiple sclerosis and rheumatoid arthritis), respectively. Applicants also show the failure to take up PEG-HCCs leaves major functions of macrophages intact. Such results suggest that the selective activity of PEG-HCCs can be utilized to treat T cell-mediated autoimmune and inflammatory diseases without inducing generalized immunosuppression.

PEG-HCCs are advantageous over existing antioxidants in that they preferentially scavenge SO and hydroxyl radicals, exhibit potent yet selective antioxidant activity, do not react with nitric oxide, do not pass radicals onto other molecules, are bioavailable, exhibit low toxicity in rodents, and do not rapidly inactivate. For instance, in studies of superoxide (SO) quenching by electron paramagnetic resonance spectroscopy, 70 μg of PEG-HCCs had a quenching effect similar to that of 10 U/mg superoxide dismutase. This value is similar to the total superoxide dismutase activity measured in a whole rat brain, which is 13 U/mg protein. The value is also higher than the value for superoxide dismutase activity reported from post-mortem human spinal cords, which ranges between 4 and 6 U/mg protein. PEG-HCCs are also advantageous because they can be utilized as nanovectors that can be used to deliver small molecule drugs to biological locations of interest.

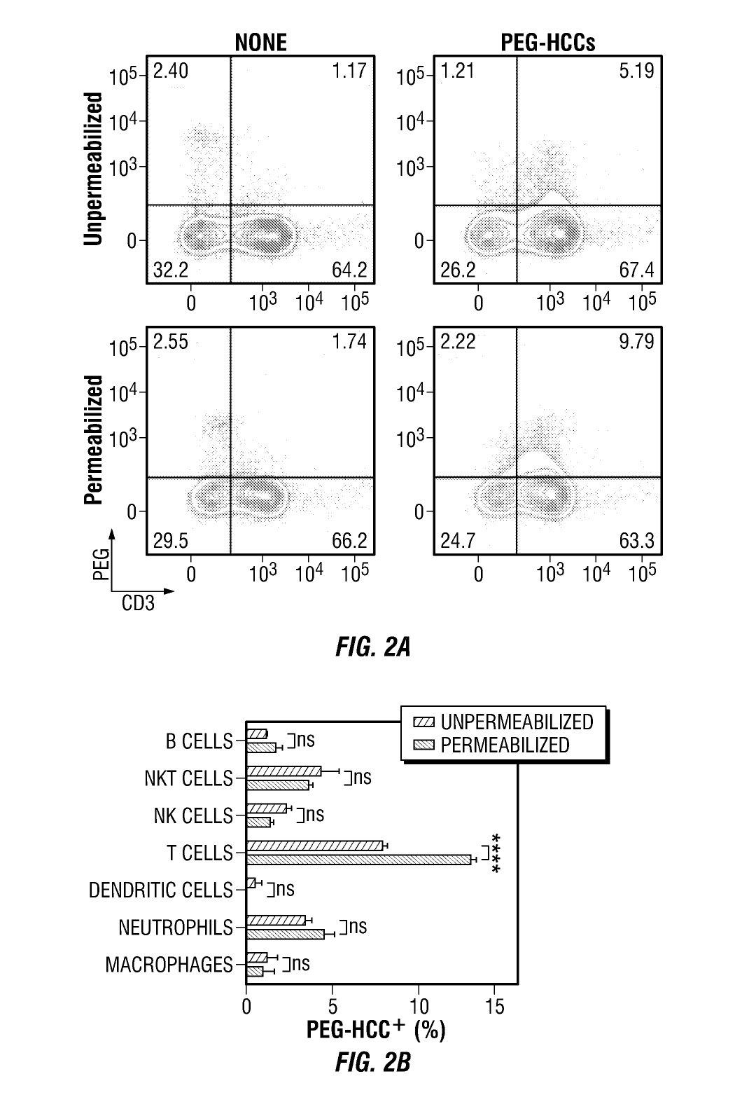

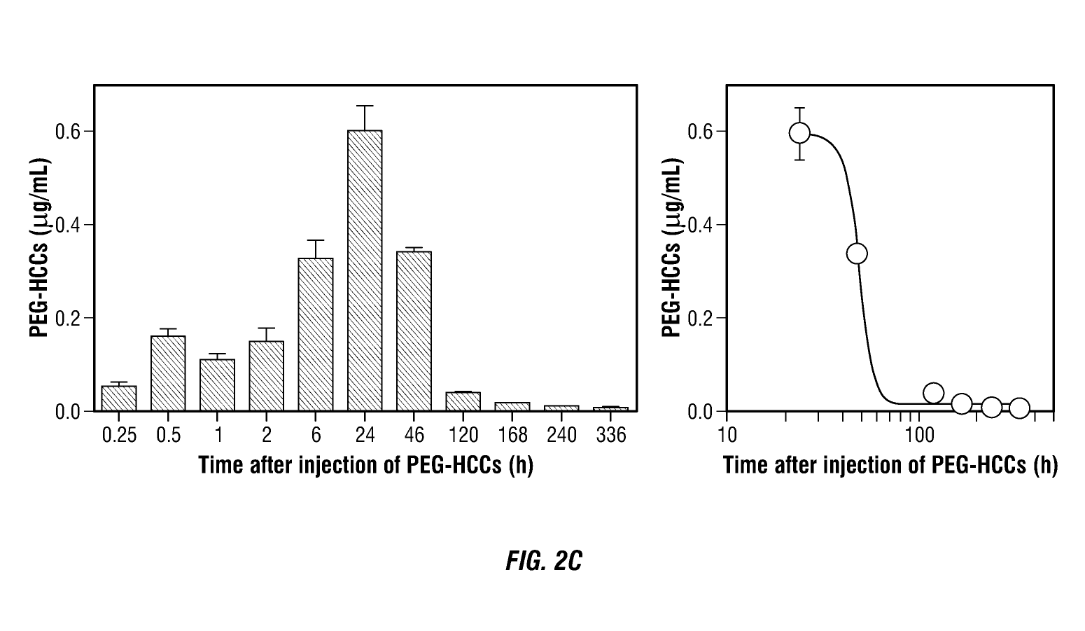

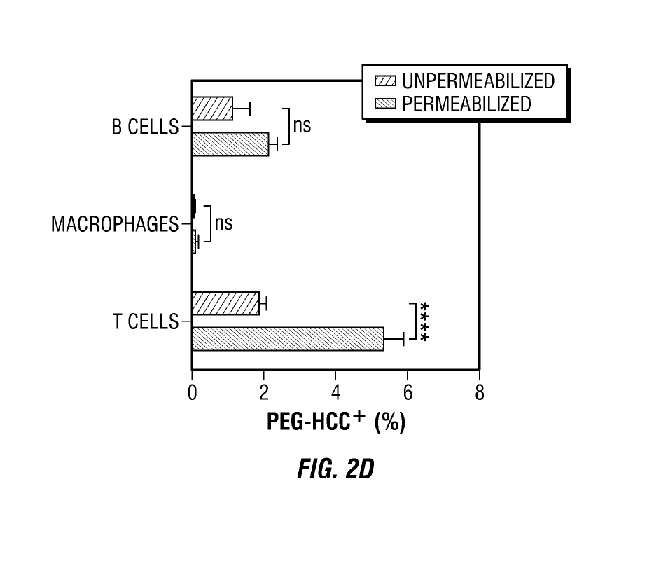

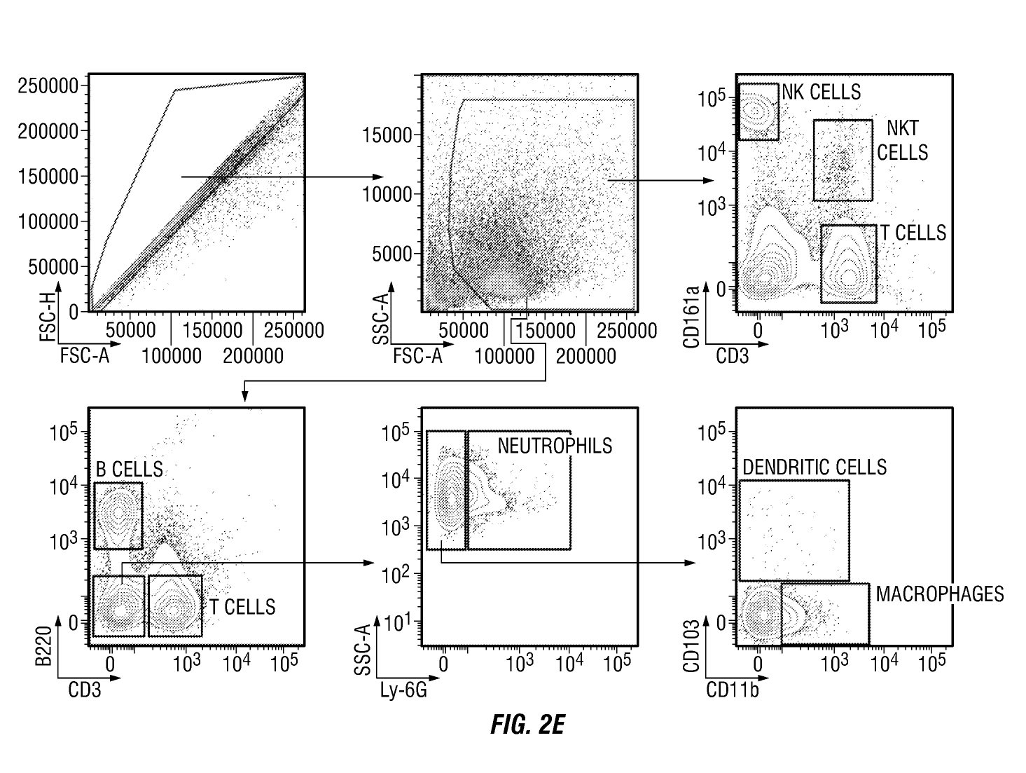

Applicants investigated whether PEG-HCCs enter major immune cell populations in the spleen to determine if they will be in contact with intracellular superoxide radicals (SO). Using flow cytometry (FCM), Applicants found that primary rat splenocytes incubated with the nanoparticles exhibited an increased PEG-HCC signal upon cell permeabilization, indicating that the nanoparticles were internalized and not just bound to the cell surface (FIG. 2A). Moreover, such an effect was more apparent in CD3⁺ cells, suggesting that PEG-HCCs are preferentially internalized by T cells (FIG. 2A).

Previous studies have shown that PEG-HCCs can enter other cell types. Therefore, Applicants assessed the uptake of PEG-HCCs by various cells, such as CD3⁻ splenocytes. In particular, Applicants assessed the uptake of PEG-HCCs into splenic B cells (CD3⁻B220⁺), neutrophils (CD3⁻B220⁻Ly-6G⁺), macrophages (CD3⁻B220⁻Ly-6G⁻CD103⁻CD11b⁺), dendritic cells (CD3⁻B220⁻Ly-6G⁻CD103⁺), NK cells (CD3⁻CD161a⁺) and NKT (CD3⁺CD161a⁺) cells. Applicants unexpectedly observed that the permeabilization of macrophages, B cells, NK cells, NKT cells, dendritic cells and neutrophils did not increase PEG-HCC signals (FIG. 2B). Such observations indicate that T cells selectively uptake PEG-HCCs.

Prior to ascertaining if PEG-HCCs are also preferentially internalized by T cells in vivo, Applicants determined the bioavailability of the PEG-HCCs in rat serum by enzyme-linked immunosorbent assay (ELISA) after a single subcutaneous injection of 2 mg/kg at the scruff of the neck (FIG. 2C). Applicants showed that subcutaneous delivery markedly enhances the half-life to 25 hours (FIG. 2C). PEG-HCCs also reached maximal levels in serum 24 hours after injection, likely due to the formation of a slow-release depot beneath the skin.

Utilizing the results from the pharmacokinetic study, Applicants then injected rats subcutaneously with 2 mg/kg of PEG-HCCs, isolated splenocytes after 24 hours, and evaluated the uptake of PEG-HCCs by various cells. The splenocytes were collected 24 hours later and stained with antibodies directed to CD3, CD4, CD11b/c, and B220. The splenocytes were then permeabilized for detection of both intracellular and extracellular PEG-HCCs or left intact to detect extracellular PEG-HCCs. Applicants found that the PEG-HCCs continue to have an exquisite capacity to enter T cells (CD3⁺B220⁻) over macrophages (CD3⁻CD11b/c⁺CD4⁺) and B cells (CD3⁻3220⁺) (FIG. 2D). Such results corroborate the in vitro findings.

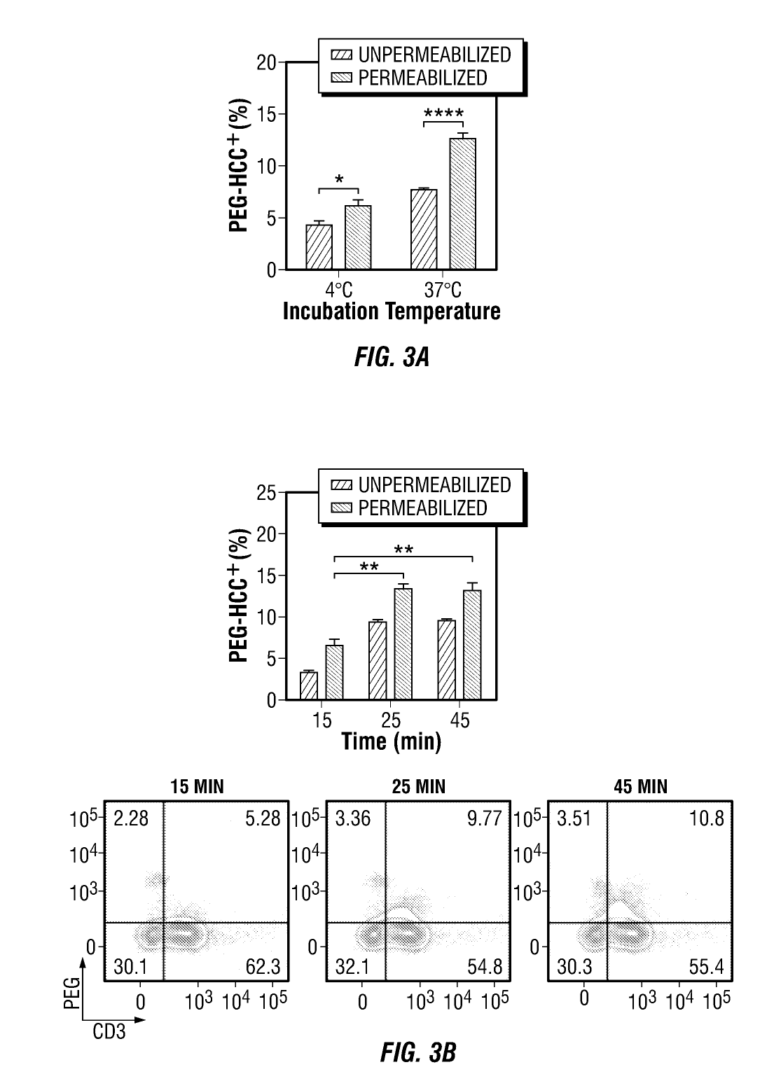

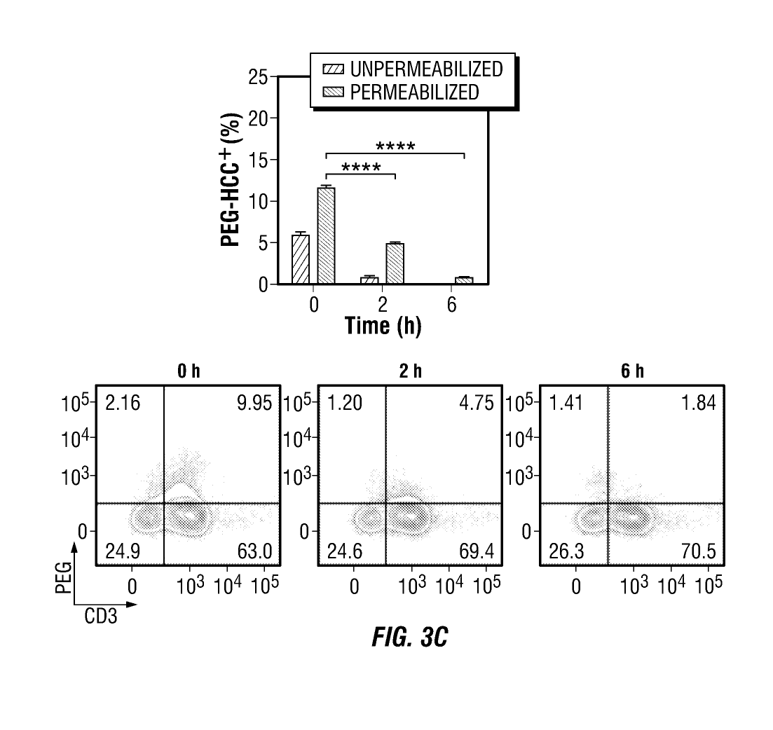

To evaluate the effect of endocytosis-inhibiting conditions on the uptake by T cells, PEG-HCCs were incubated at 4° C. and analyzed by FCM. Applicants found that such conditions attenuate, but do not prevent internalization (FIG. 3A). Without being bound by theory, such results suggest that PEG-HCC uptake occurs mainly via endocytosis.

Next, Applicants examined the kinetics of PEG-HCC influx into T cells and found that they reach maximal intracellular levels after 25 minutes of incubation (FIG. 3B). In addition, Applicants found that PEG-HCCs leave T cells gradually and become nearly undetectable after 6 hours (FIG. 3C). Without being bound by theory, such results suggest that PEG-HCCs do not accumulate inside cells.

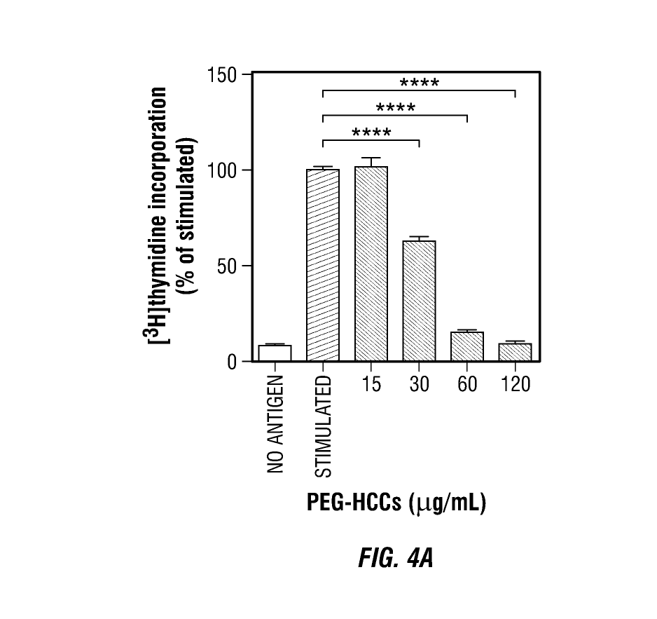

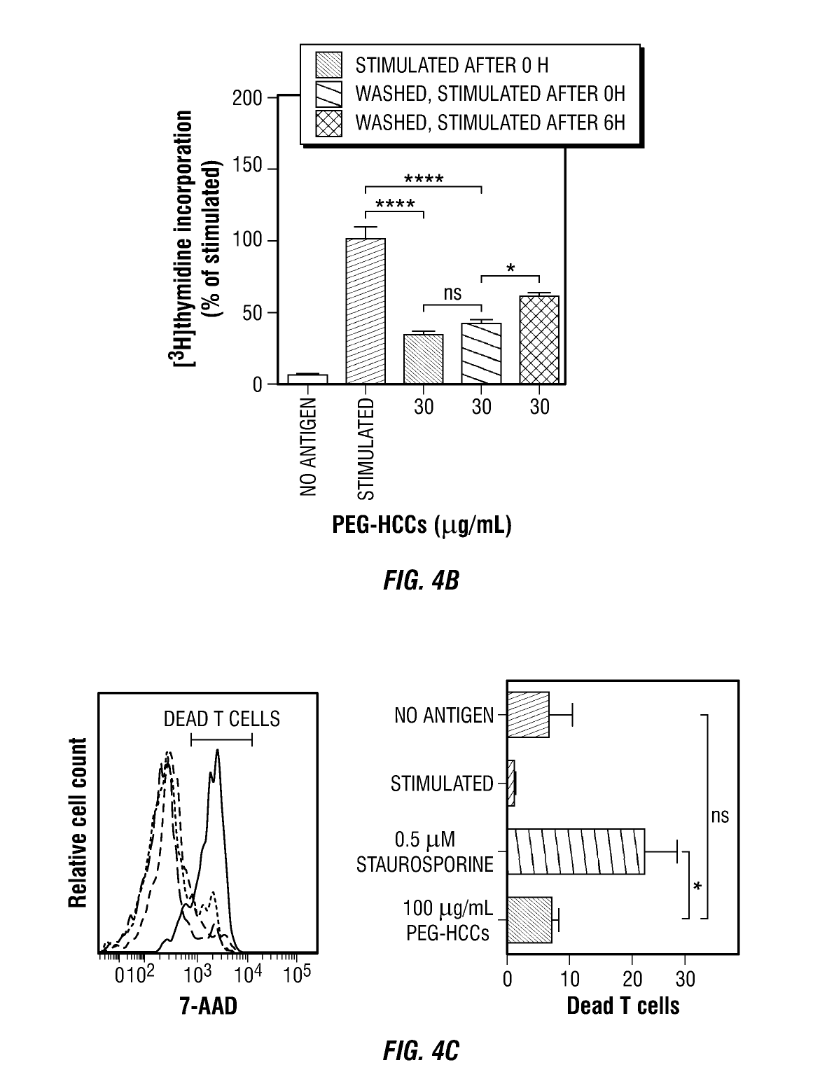

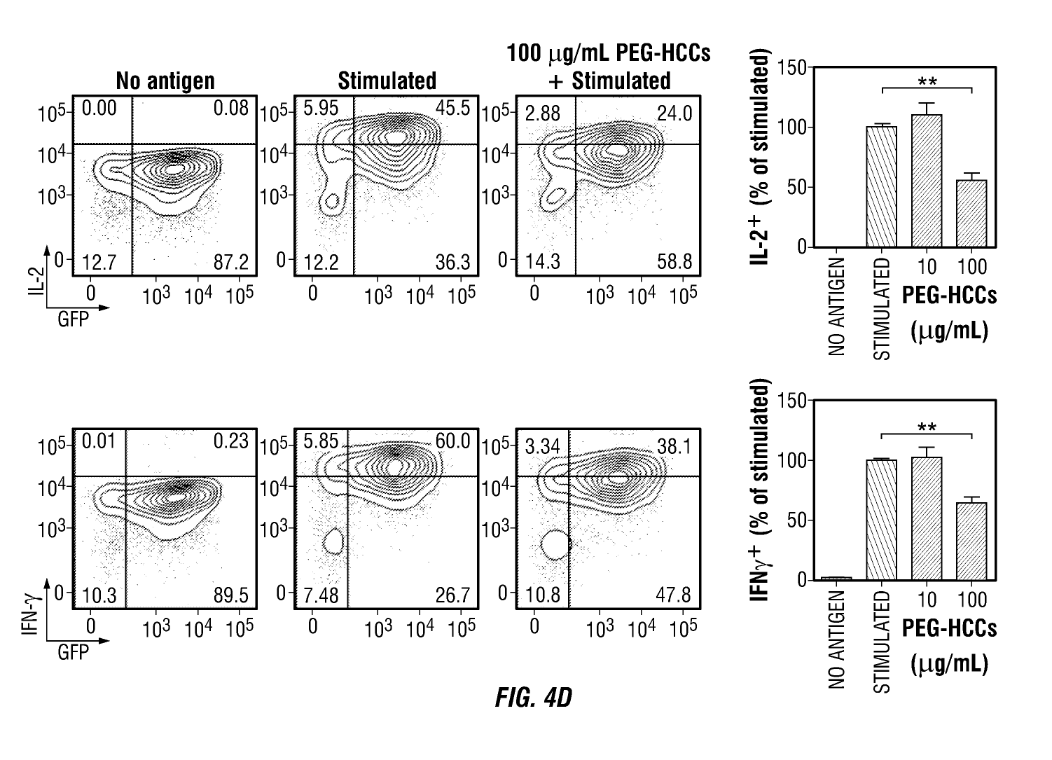

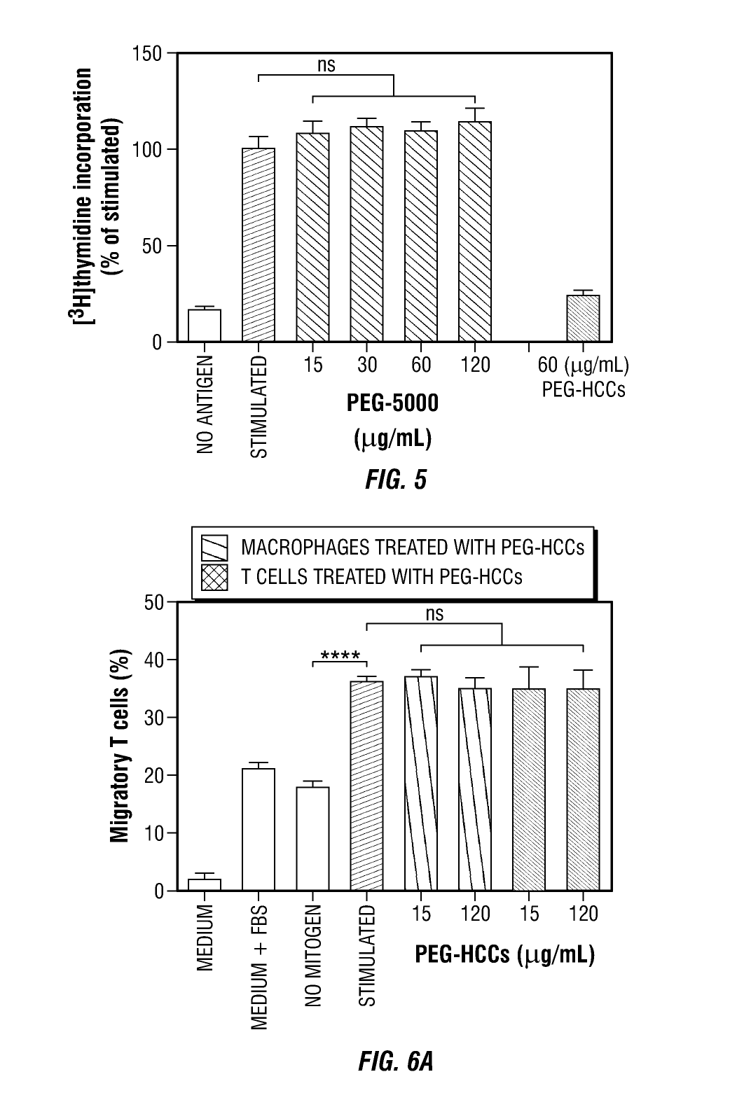

In addition, Applicants assessed the consequences of PEG-HCC internalization on the cellular activity of T cells, the predominant cell type responsible for autoimmune disease. When Applicants incubated primary GFP-transduced ovalbumin-specific rat T cells (CD4⁺CCR7⁻ CD45RC⁻Kv1.3^{high}) with PEG-HCCs and stimulated the cells with ovalbumin, Applicants found a dose-dependent reduction in both intracellular SO levels and proliferation (FIG. 4A). However, the decrease in T cell proliferation was not due to the presence of PEG, which alone was not sufficient to induce an inhibitory response (FIG. 5). In addition, washing away excess PEG-HCCs and immediately stimulating the cells did not alter the effect on proliferation, confirming that PEG-HCCs need to be internalized to alter T cell activity (FIG. 4B).

In contrast, stimulating cells after 6 hours rescued the inhibitory effect on proliferation, (FIG. 4B). This result is in alignment with the kinetics of nanoparticle loss and suggests that PEG-HCCs have a reversible effect on T cell activity.

To investigate whether the observed effect on T cell proliferation was attributed to a cytotoxic effect by the nanoparticles, Applicants utilized FCM to analyze cell death in T cells treated with PEG-HCCs prior to stimulation and found that they did not prompt any changes in cell viability (FIG. 4C). Applicants also utilized FCM analysis to examine the effects of PEG-HCCs on the production of pro-inflammatory cytokines in T cells stimulated by ovalbumin and found a ˜30% reduction in the levels of interleukin (IL)-2 and interferon (IFN)-γ (FIG. 4D).

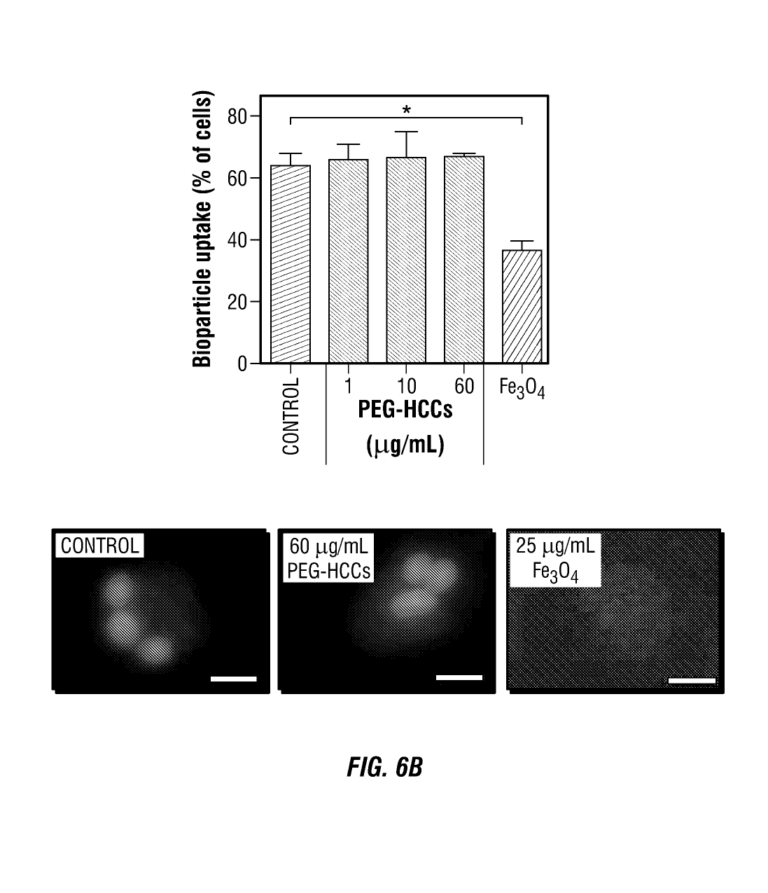

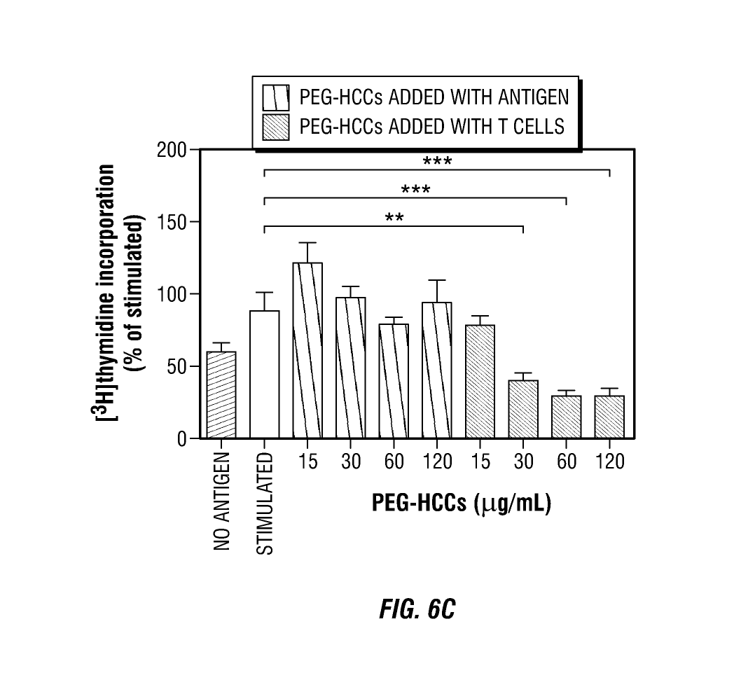

While Applicants demonstrated that macrophages do not internalize PEG-HCCs, Applicants investigated whether the observed effects on T cell activity by PEG-HCCs stemmed from an alteration in function of antigen-presenting cells, which include macrophages. Applicants found no effect on T cell migration across transwell filters towards supernatant collected from the culture of primary rat intra-peritoneal macrophages that were treated with PEG-HCCs prior to stimulation with lipopolysaccharide (LPS) (FIG. 6A). This result indicates that PEG-HCCs do not affect the production of chemo-attractants by macrophages.

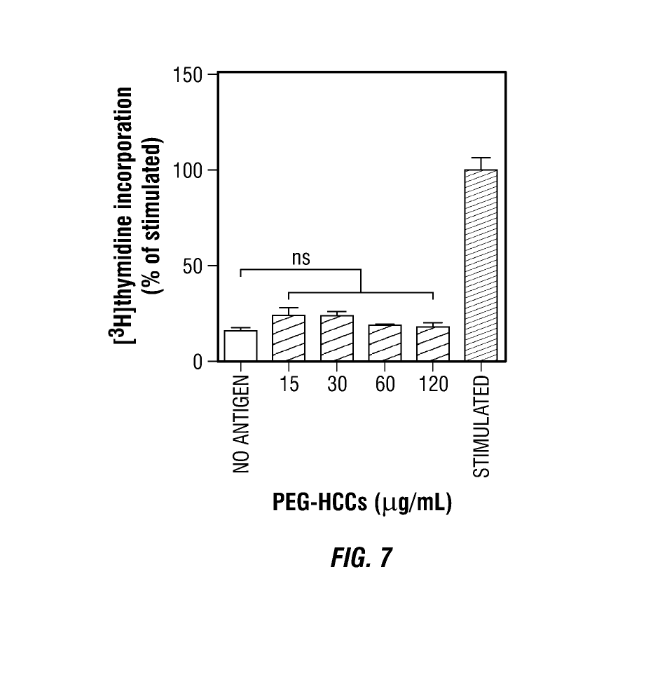

In addition, treating T cells with PEG-HCCs did not affect their migration (FIG. 6A). Such results indicate that PEG-HCCs have no effect on the proliferation of unstimulated T cells (FIG. 7).

Next, Applicants found that phagocytosis of zymosan bioparticles was unaltered when macrophages were incubated with PEG-HCCs (FIG. 6B), unlike other nanoparticles. Finally, when macrophages were treated with PEG-HCCs before being loaded with ovalbumin to provide ovalbumin-specific T cells, Applicants found that there was no effect on T cell proliferation (FIG. 6C). However, the addition of PEG-HCCs to macrophages at the same time as the T cells led to a reduction in T cell proliferation (FIG. 6C), similar to findings in FIG. 4A. Such results indicate that PEG-HCCs do not modify antigen processing and presentation by macrophages.

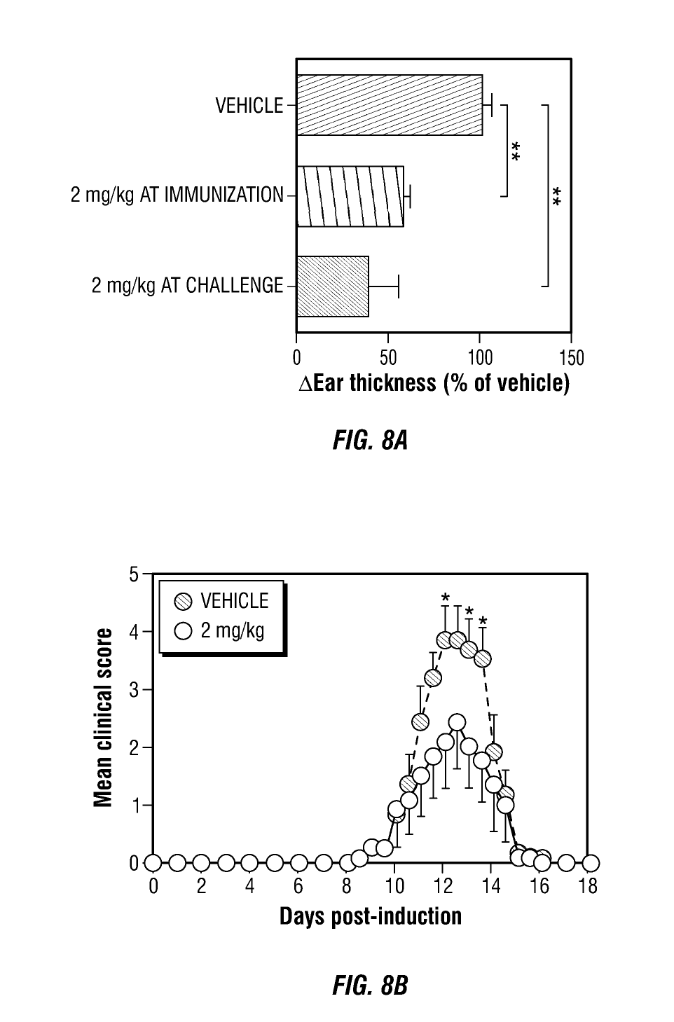

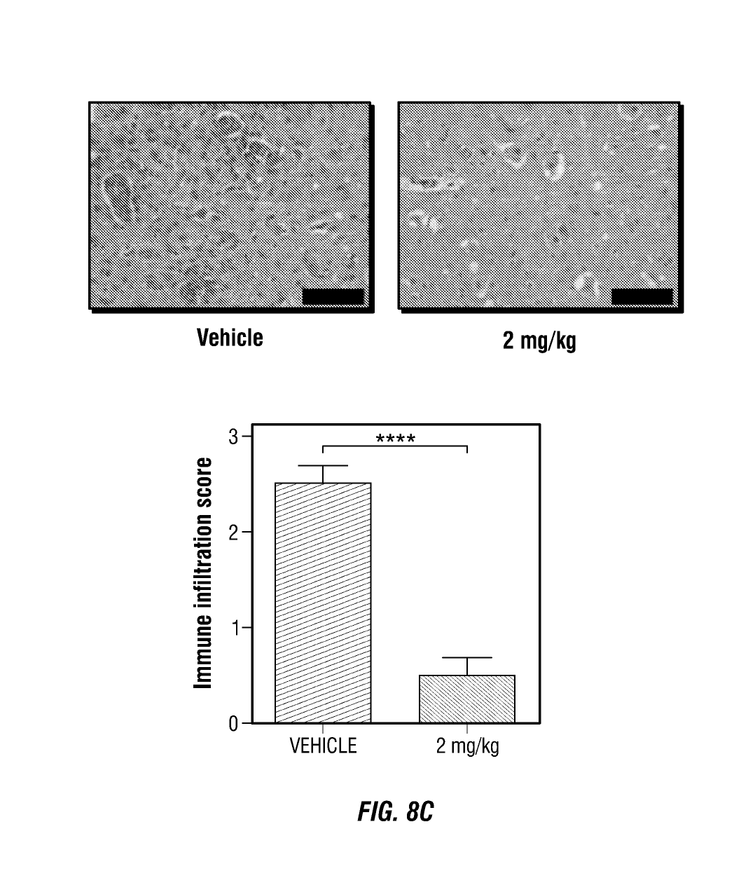

Next, Applicants examined the effects of PEG-HCCs on animal disease models that are mediated by T cells. Applicants elicited an active delayed-type hypersensitivity response (DTH) against ovalbumin in the ears of rats and found that a single subcutaneous injection of 2 mg kg⁻¹ PEG-HCCs either at the time of immunization or challenge was sufficient to decrease inflammation (FIG. 8A). This finding prompted Applicants to test the effect of PEG-HCCs on rats with myelin basic protein-induced EAE. Applicants found that the subcutaneous treatment of rats with 2 mg/kg of PEG-HCCs every three days starting at the onset of disease signs significantly reduced clinical scores (FIG. 8B). Histologic analysis of spinal cords isolated from EAE rats at the peak of disease revealed a decrease in inflammatory foci, indicating decreased infiltration of immune cells into the spinal cord (FIG. 8C).

In this Example, Applicants demonstrated that PEG-HCCs are selective immunomodulators that can be utilized to treat inflammatory diseases. Applicants established that PEG-HCCs are preferentially internalized by T cells over other immune cells.

While Applicants were not able to identify a single mechanism responsible for T cell uptake by PEG-HCCs, Applicants' data indicate that PEG-HCCs enter principally via endocytosis. Applicants also demonstrate that PEG-HCC uptake by T cells can also inhibit the production of pro-inflammatory cytokines and T cell proliferation without having a permanent or cytotoxic effect on the T cells. Such findings are in line with studies demonstrating the use of antioxidants to attenuate T cell activation induced by mitogens or antigens.

Furthermore, results observed on T cell activity by PEG-HCCs were not due to an extraneous effect on chemo-attraction, phagocytosis and antigen processing and presentation by macrophages, which are essential steps for the physiological activation of T cells. A major implication of these data is that, by failing to internalize PEG-HCCs, key functions of macrophages remain unaltered. This demonstrates that PEG-HCCs comprise a strategic selectivity absent in established treatments of autoimmune disease. This also suggests treatment with PEG-HCCs will not induce generalized immunosuppression.

In addition, the significance of Applicants' in vitro results on T cell activity by PEG-HCCs was clearly demonstrated by the findings that administration of these nanoparticles into rat models lead to a reduction in DTH inflammation, EAE scores and immune infiltration into the spinal cord. Together these data suggest that PEG-HCCs are an invaluable tool for treating T cell-mediated inflammatory diseases (e.g., T cell-mediated autoimmune diseases).

### Example 2

### PEG-HCCs Enter Human T Cells

In this Example, Applicants provide additional data to demonstrate that PEG-HCCs preferentially enter human T cells. Such results further affirm and supplement the results provided in Example 1.

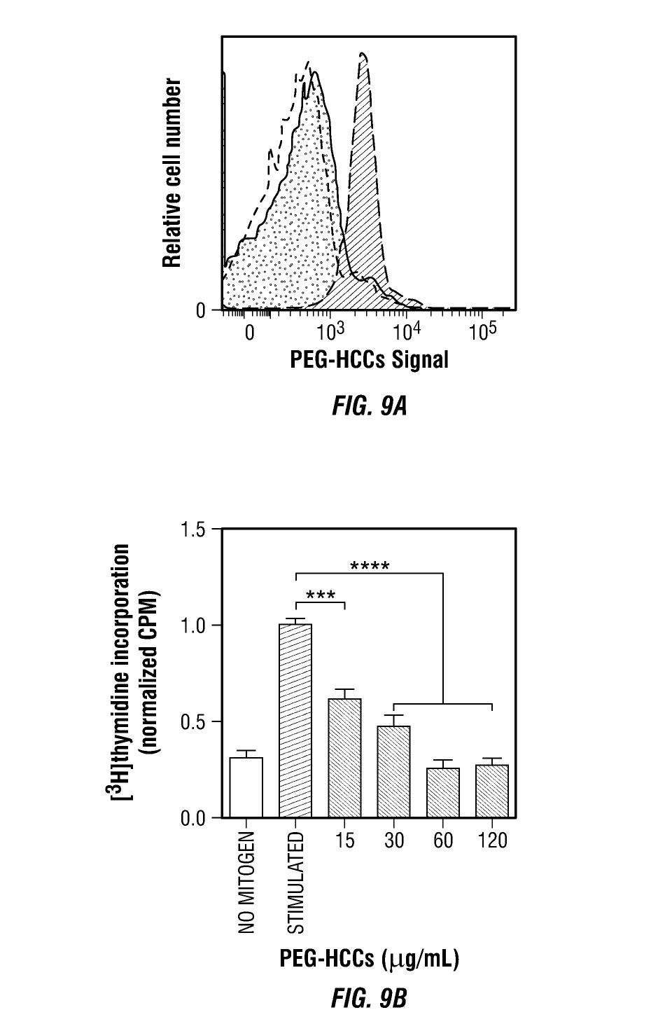

Applicants used flow cytometry to detect PEG-HCCs at the surface of non-permeabilized T cells and inside permeabilized T cells. As shown in FIG. 9A, Applicants found that the majority of T cell-associated PEG-HCCs after 10 minutes of incubation at 37° C. were intracellular. These results demonstrate that PEG-HCCs are in contact with intracellular superoxide. Moreover, as shown in FIG. 9B, a reduction in the proliferation of stimulated human T cells was observed upon internalization of PEG-HCCs into human T cells.

### Example 3

### Effect of PEG-HCCs in Animal Models of T Lymphocyte-Mediated Autoimmune Disease

In this Example, Applicants provide additional data regarding the effects of PEG-HCCs in animal models of T cell-mediated autoimmune diseases. Such results further affirm and supplement the results provided in Examples 1 and 2.

### Example 3.1

### PEG-HCCs Reduce the Number of Lesions to the Blood-Brain Barrier in an Active Acute Model of Multiple Sclerosis in Rats

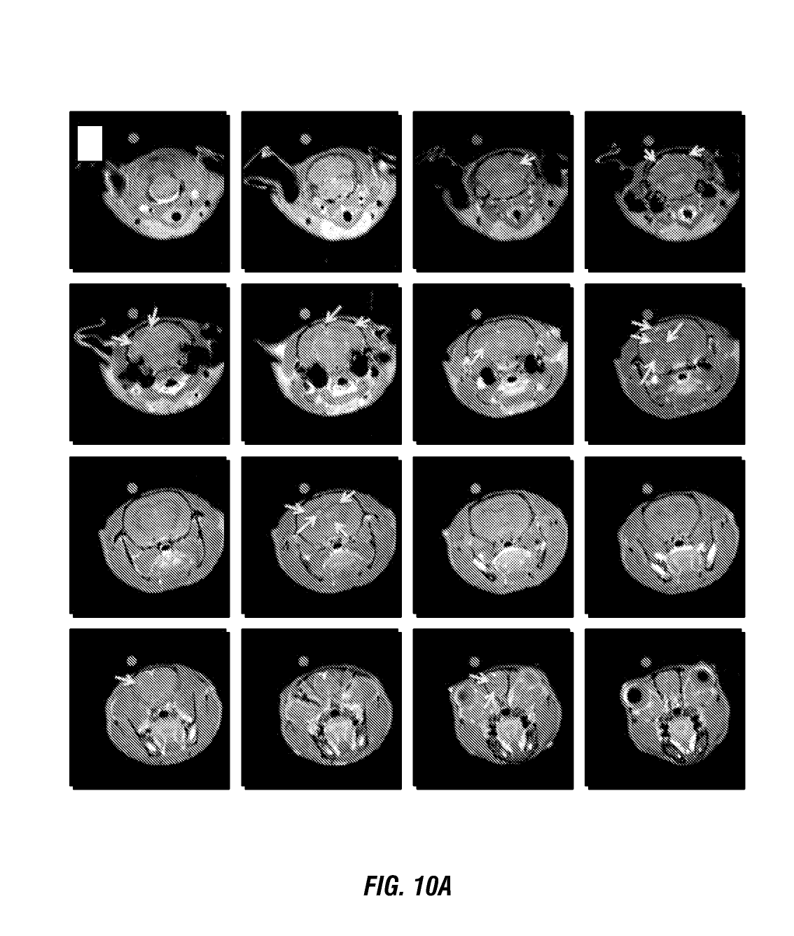

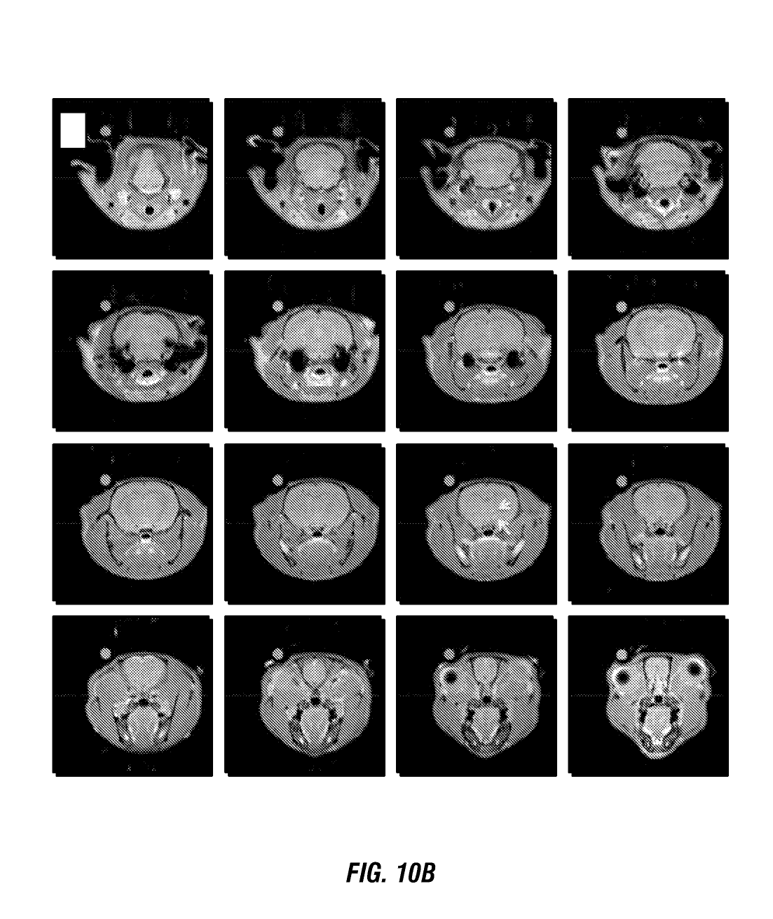

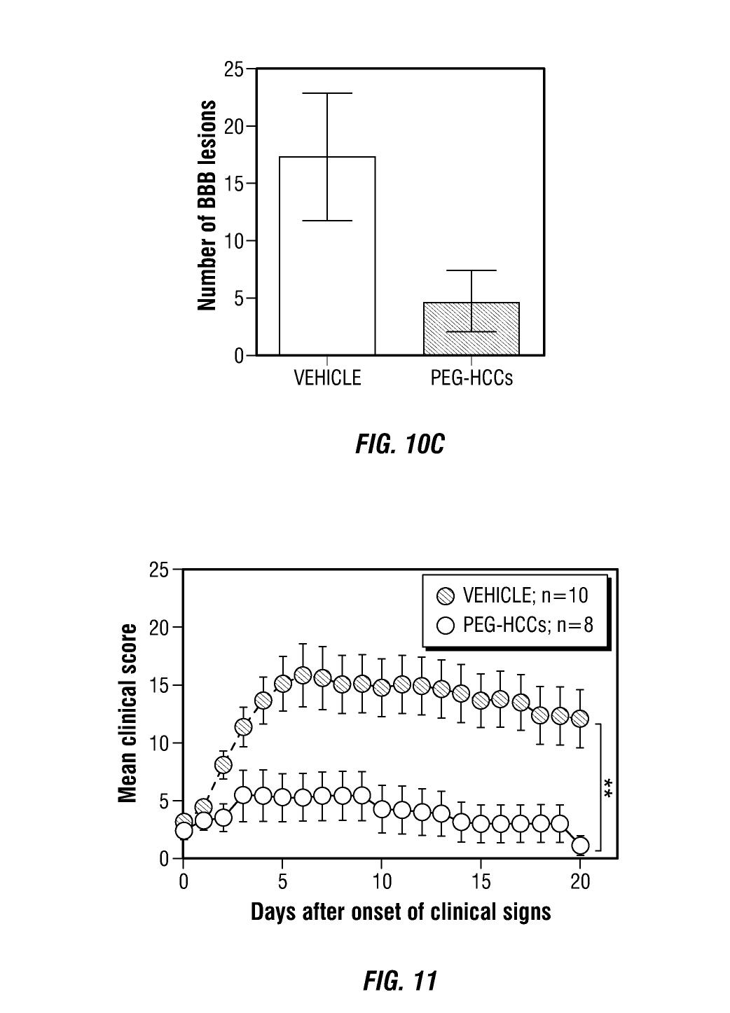

One way to detect central nervous system lesions preclinically and clinically is to use dynamic contrast enhanced (DCE) MRI imaging. In this method, a chelated Gd³⁺ contrast agent is introduced intraveneously, resulting in positive contrast enhancement at the lesion sites. In this case, Applicants performed DCE MRI imaging on active acute models of EAE induced by immunization of Lewis rats against myelin-basic protein in complete Freund's adjuvant. Rats were treated with vehicle or PEG-HCCs at the time of immunization and 7 days later (FIG. 10). The first panel (FIG. 10A) depicts images acquired of the rat with a model of multiple sclerosis treated with vehicle. The yellow arrows point to the lesion enhancing areas. The second panel (FIG. 10B) depicts images acquired of a rat with a model of multiple sclerosis treated with PEG-HCCs. Note the marked reduction in lesion enhancing areas. The chart in FIG. 10C quantifies the lesions. These results show that PEG-HCCs can reduce the number of lesions to the blood-brain barrier in a model of multiple sclerosis in rats.

### Example 3.2

### PEG-HCCs Prevent a Delayed Type Hypersensitivity (DTH) Reaction in Rats and Reduce Disease Severity in a Rat Model of Rheumatoid Arthritis

An active DTH reaction was elicited against ovalbumin as described (Mol Pharmacol 2005; 67:1369-1381; J Vis Exp 2007; 6:e237; J Vis Exp 2007; 8:e325; J Biol Chem 2008; 283:988-997; and J Pharmacol Exp Ther 2012; 342:642-653). A single subcutaneous administration of PEG-HCCs, at time of immunization or challenge, significantly reduced ear swelling, a measure of T cell-mediated inflammation (see, e.g., FIG. 8A in Example 1). Pristane-induced arthritis, an animal model of rheumatoid arthritis, was induced and monitored in rats, as described. Applicants found that the administration of PEG-HCCs every four days starting at the onset of clinical signs significantly reduced disease severity (FIG. 11). These results demonstrate that PEG-HCCs can inhibit T cell-mediated immune reactions in vivo.

### Example 3.3

### PEG-HCCs Showed a Trend Towards Reducing R-EAE Clinical Scores During the Relapsing Phase of Disease

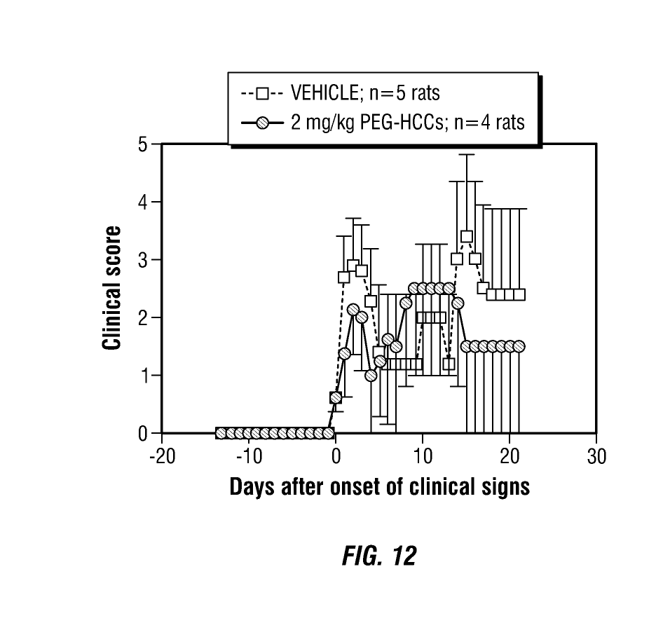

In a small trial, Applicants induced R-EAE in a small cohort of DA rats (n=9 rats split into 2 treatment groups) and did a prevention trial with PEG-HCCs. PEG-HCCs displayed a minor effect on the first episode of disease (FIG. 12). Such results were unexpected.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of treating an inflammatory disease in a subject, wherein the method comprises:
administering a carbon material to the subject,
where in the carbon material selectively targets T cells in the subject.

2. The method of claim 1, wherein the carbon material is selected from the group consisting of graphene quantum dots, graphene, graphene oxide, carbon black, activated carbon, carbon nanotubes, ultra-short single-walled carbon nanotubes, and combinations thereof.

3. The method of claim 1, wherein the carbon material has a serum half-life of between about 15 hours to about 40 hours.

4. The method of claim 1, wherein the carbon material has a length ranging from about 10 nm to about 100 nm.

5. The method of claim 1, wherein the carbon material has a length ranging from about 10 nm to about 50 nm.

6. The method of claim 1, wherein the carbon material is functionalized with a plurality of functional groups.

7. The method of claim 6, wherein the functional groups are selected from the group consisting of polyethylene glycols, polypropylene glycols, poly(acrylic acid), polysaccharides, poly(alcohols), poly(vinyl alcohol), polyamines, polyethylene imines, poly(vinyl amines), ketones, esters, amides, carboxyl groups, oxides, hydroxyl groups, alkoxy groups, and combinations thereof.

8. The method of claim 6, wherein the functional groups comprise polyethylene glycols.

9. The method of claim 1, wherein carbon material comprises one or more transport moieties.

10. The method of claim 9, wherein the transport moieties are selected from the group consisting of adamantane moieties (ADM), dimethyladamantane moieties, lipophilic moieties, small molecules, cannabinoids, epi-cannabinoids, peptides, saccharides, and combinations thereof.

11. The method of claim 1, wherein carbon material is oxidized.

12. The method of claim 1, wherein the carbon material comprises carbon nanotubes.

13. The method of claim 12, wherein the carbon nanotubes are selected from the group consisting of single-walled carbon nanotubes, ultra-short single-walled carbon nanotubes, multi-walled carbon nanotubes, double-walled carbon nanotubes, and combinations thereof.

14. The method of claim 1, wherein the carbon material comprises ultra-short single-wall carbon nanotubes.

15. The method of claim 14, wherein the ultra-short single-wall carbon nanotubes are functionalized with a plurality of functional groups.

16. The method of claim 14, wherein ultra-short single-wall carbon nanotubes comprise poly(ethylene glycol)-functionalized ultra-short single-walled carbon nanotubes.

17. The method of claim 14, wherein the ultra-short single-walled carbon nanotubes have lengths ranging from about 10 nm to about 100 nm.

18. The method of claim 14, wherein the ultra-short single-walled carbon nanotubes have lengths ranging from about 10 nm to about 50 nm.

19. The method of claim 1, wherein the administering occurs by a method selected from the group consisting of oral administration, inhalation, subcutaneous administration, topical administration, transdermal administration, intra-articular administration, intravenous administration, intraperitoneal administration, intramuscular administration, intrathecal injection, sub-lingual administration, intranasal administration, and combinations thereof.

20. The method of claim 1, wherein the subject is suffering from an inflammatory disease.

21. The method of claim 20, wherein the subject is a mammal.

22. The method of claim 20, wherein the subject is a human being.

23. The method of claim 1, wherein the inflammatory disease is selected from the group consisting of chronic inflammatory diseases, autoimmune diseases, T cell-mediated diseases, T cell-mediated autoimmune diseases, T cell-mediated inflammatory diseases, multiple sclerosis, rheumatoid arthritis, reactive arthritis, ankylosing spondylitis, systemic lupus erythematosus, glomerulonephritis, psoriasis, scleroderma, alopecia aerata, type 1 diabetes mellitus, celiac sprue disease, colitis, pernicious anemia, encephalomyelitis, vasculitis, thyroiditis, Addison's disease, Sjögren's syndrome, antiphospholipid syndrome, autoimmune cardiomyopathy, autoimmune hemolytic anemia, autoimmune hepatitis, autoimmune inner ear disease, autoimmune lymphoproliferative disorder, autoimmune peripheral neuropathy, pancreatitis, polyendocrine syndrome, thrombocytopenic purpura, uveitis, Behcet's disease, narcolepsy, myositis, polychondritis, asthma, chronic obstructive pulmonary disease, graft-versus-host disease, chronic graft rejection, and combinations thereof.

24. The method of claim 1, wherein the administering of the carbon material comprises daily administration.

25. The method of claim 24, wherein the daily administration lasts from about 3 days to about 3 months.

26. The method of claim 24, wherein the daily administration comprises from about 1 carbon material administration per day to about 5 carbon material administrations per day.

27. The method of claim 1, wherein the administering comprises carbon material administration at dosages that range from about 1 mg/kg of the subject's weight to about 5 mg/kg of the subject's weight.

28. The method of claim 1, wherein the administering of the carbon material reduces or inhibits T cell-mediated reactions in the subject.

29. The method of claim 1, wherein the carbon material selectively targets T cells over other types of immune cells.

30. The method of claim 1, wherein the carbon material selectively targets T cells by preferential uptake into the T cells.

31. The method of claim 1, wherein the carbon material reduces or inhibits proliferation of targeted T cells.

32. The method of claim 1, wherein the carbon material reduces or inhibits cytokine production by targeted T cells.

33. The method of claim 1, wherein the carbon material reduces or inhibits T-cell signaling by targeted T cells.

34. The method of claim 1, wherein the carbon material reduces intracellular oxidant content in targeted T cells.

35. The method of claim 1, wherein carbon material does not induce apoptosis in targeted T cells.

36. A method of modulating T cells, wherein the method comprises incubating the T cells with a carbon material.

37. The method of claim 36, wherein the carbon material is selected from the group consisting of graphene quantum dots, graphene, graphene oxide, carbon black, activated carbon, carbon nanotubes, ultra-short single-walled carbon nanotubes, and combinations thereof.

38. The method of claim 36, wherein the carbon material is functionalized with a plurality of functional groups.

39. The method of claim 38, wherein the functional groups are selected from the group consisting of polyethylene glycols, polypropylene glycols, poly(acrylic acid), polysaccharides, poly(alcohols), poly(vinyl alcohol), polyamines, polyethylene imines, poly(vinyl amines), ketones, esters, amides, carboxyl groups, oxides, hydroxyl groups, alkoxy groups, and combinations thereof.

40. The method of claim 39, wherein the functional groups comprise polyethylene glycols.

41. The method of claim 36, wherein carbon material comprises one or more transport moieties.

42. The method of claim 41, wherein the transport moieties are selected from the group consisting of adamantane moieties (ADM), dimethyladamantane moieties, lipophilic moieties, small molecules, cannabinoids, epi-cannabinoids, peptides, saccharides, and combinations thereof.

43. The method of claim 36, wherein the carbon material comprises ultra-short single-wall carbon nanotubes.

44. The method of claim 43, wherein the ultra-short single-wall carbon nanotubes are functionalized with a plurality of functional groups.

45. The method of claim 43, wherein ultra-short single-wall carbon nanotubes comprise poly(ethylene glycol)-functionalized ultra-short single-walled carbon nanotubes.

46. The method of claim 43, wherein the ultra-short single-walled carbon nanotubes have lengths ranging from about 10 nm to about 50 nm.

47. The method of claim 36, wherein the carbon material reduces or inhibits T-cell mediated reactions.

48. The method of claim 36, wherein the method occurs ex-vivo.

49. The method of claim 36, wherein the method occurs ex-vivo in the presence of other types of immune cells.

50. The method of claim 36, wherein the carbon material selectively targets T cells over other types of immune cells.

51. The method of claim 36, wherein the carbon material selectively targets T cells by preferential uptake into the T cells.

52. The method of claim 36, wherein the carbon material reduces or inhibits proliferation of targeted T cells.

53. The method of claim 36, wherein the carbon material reduces or inhibits cytokine production by targeted T cells.

54. The method of claim 36, wherein the carbon material reduces or inhibits T cell signaling by targeted T cells.

55. The method of claim 36, wherein the carbon material reduces intracellular oxidant content in targeted T cells.

56. The method of claim 36, wherein carbon material does not induce apoptosis in targeted T cells.
